(19) United States
(12) Patent Application Publication
(10) Pub. No.: US 2020/0383327 A1
(43) Pub. Date: Dec. 10, 2020

(54) METHOD OF IMPROVING PLANT PERFORMANCE

(71) Applicant: The State of Israel, Ministry of Agriculture & Rural Development, Agricultural Research Organization, Beit-Dagan (IL)

(72) Inventors: Michal OREN-SHAMIR, Rehovot (IL); Moran OLIVA, Mazkeret Batia (IL); Efraim LEWINSOHN, Timrat (IL); Noam ALKAN, Moshav Kidron (IL); Yigal ELAD, Ganei-Tikva (IL)

(21) Appl. No.: 17/002,370

(22) Filed: Aug. 25, 2020

Related U.S. Application Data
(63) Continuation-in-part of application No. PCT/IL2019/050218, filed on Feb. 26, 2020.
(60) Provisional application No. 62/634,992, filed on Feb. 26, 2018.
(60) Provisional application No. 62/794,629, filed on Jan. 20, 2019.

(51) Int. Cl. A01N 37/44

(52) U.S. Cl. CPC A01N 37/44

(57) ABSTRACT

Provided composition and methods for controlling, treating or preventing a pathogenic infection in a plant with a phenylalanine solution. The plant may be at a post-harvest and/or at a pre-harvest stage.

### CROSS REFERENCE TO RELATED APPLICATIONS

This application is a Continuation-In-Part of PCT Application No. PCT/IL2019/050218, filed Feb. 26, 2019, which claims the benefit of priority of U.S. Provisional Patent Application No. 62/634,992 filed Feb. 26, 2018, and U.S. Provisional Patent Application No. 62/794,629 filed Jan. 20, 2019, the contents of which are all incorporated herein by reference in their entirety.

### FIELD OF THE INVENTION

The present invention, in some embodiments thereof, relates to a method of improving plant performance, e.g., growth parameters, crop, and pathogenic resistance.

### BACKGROUND OF THE INVENTION

Damage to plants can be caused by biotic and abiotic stressors. Biotic stressors include arthropods, fungi, viruses, bacteria, moths and nematodes.

Plant disease outbreaks have resulted in catastrophic crop failures that have triggered famines and caused major social change. Plant diseases affect yield quantity and quality. Generally, the best strategy for plant disease control is to use resistant cultivars selected or developed by plant breeders for this purpose. However, the potential for serious crop disease epidemics persists today.

Plants use a wide range of defense mechanisms to avoid infection by pathogens and attack by parasites. These include local induced defense response, formation of local lesions with increased production of reactive oxygen species (ROS), formation of antimicrobial phenolic compounds, deposition of callose and lignin, and induction of pathogenesis related (PR) protein synthesis (Lattanzio et al., 2006). Enhancement in phenol phytoalexins and other aromatic anti-oxidant compounds following biotic stress is the result of the induction of the shikimate pathway synthesizing aromatic amino acids (AAAs) and of downstream specific polyphenol pathways (Pandey et al., 2015; Camañes et al., 2015).

Postharvest losses in plants can be either quantitative or qualitative. Even though emphasis in crop research nowadays is increasing shifting from quantity to production quality, there is still little improvement in the quality of commercially produced plant varieties, hence resulting in high quality losses.

Global regulatory requirements are becoming more and more demanding with respect to the use of pesticides, particularly unmanaged or unnecessary pesticide residues. In addition, the general public would rather consume chemical-free (e.g., less pesticides residue) fruits and vegetables. A particular consequence of this is that there is an increasing need to have more efficient yet safe methods of protecting plants, and products thereof, such as but not limited to fruits and vegetables, from pathogens.

### SUMMARY OF THE INVENTION

According to one aspect, there is provided a method of controlling a pathogenic infection in a plant, comprising contacting the plant with an effective amount of phenylalanine or an analog thereof at a concentration of at least 2 mM, thereby controlling a pathogenic infection in a plant.

In some embodiments, the pathogenic infection excludes a fungal infection.

In some embodiments, the pathogenic infection is a viral infection, a bacterium infection, a moth infection, an arachnid infection, or any combination thereof

In some embodiments, the pathogenic infection is a Pseudomonas infection.

According to another aspect, there is provided a method of controlling a viral infection in a plant, comprising contacting the plant with an effective amount of a phenylalanine or an analog thereof, thereby controlling a viral infection in a plant.

According to another aspect, there is provided a method for prolonging the shelf life of a plant, the post-harvest quality of a plant, or a combination thereof, comprising the steps of: (a) pre-harvest contacting the plant with an effective amount of phenylalanine or an analog thereof (b) post-harvest contacting the plant with an effective amount of phenylalanine or an analog thereof; or (c) the combination of (a) and (b), thereby prolonging the shelf life of a plant, the post-harvest quality of a plant, or a combination thereof.

In some embodiments, the viral infection comprises a tomato brown rugose fruit virus (TBRFV).

In some embodiments, the concentration of phenylalanine or an analog is above 2 mM.

In some embodiments, contacting comprises pre-harvest contacting, post-harvest contacting or a combination thereof.

In some embodiments, contacting is when the plant is at: a post-blossom stage, a blossom stage, a pre-blossom stage, or any combination thereof.

In some embodiments, the plant is a crop.

In some embodiments, phenylalanine or an analog is formulated in a composition selected from the group consisting of: a dip, a spray, a seed coating, a concentrate, or any combination thereof

In some embodiments, contacting is contacting in the vicinity of or onto: a root, a stem, a trunk, a seed, a fruit, a flower, a leave, or any combination thereof.

In some embodiments, contacting is irrigating, drenching, dipping, soaking, injecting, coating, spraying, or any combination thereof.

In some embodiments, contacting is repeated at least twice.

In some embodiments, contacting is pre-infection, post infection, or a combination thereof.

In some embodiments, contacting is in: a storage facility, a greenhouse, an open field, or any combination thereof

According to another aspect, there is provided a method for increasing a plant performance parameter, comprising contacting the plant with an effective amount of phenylalanine or an analog thereof, thereby increasing the plant performance parameter.

In some embodiments, the performance parameter is selected from the group consisting of: plant growth, crop yield, abiotic stress resistance, and any combination thereof.

In some embodiments, growth comprises one or more parameters selected from the group consisting of: growth rate, plant weight, plant height, leaf length, leaflet area, number of nodes, and distance between adjacent nodes.

In some embodiments, the abiotic stress is suboptimal temperatures.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1C are images and graphs showing the effect of 0-12 mM (e.g., 4 mM, and 8 mM) of phenylalanine (Phe) applied by drenching or spraying on the severity of bacterial speck in tomato, 13-14 days post treatment. FIG. 1A—an image of tomato bacterial speck on a tomato leaflet caused by the bacterium Pseudomonas syringae pv. tomato. FIG. 1B is a vertical bar graph showing severity of bacterial speck on tomato plants treated with drench or spray of 4 mM solution phenylalanine. Disease was evaluated on a 0-100% severity of symptoms coverage 13 days after treatment. Bars=Standard Errors; Columns followed by a different letter are significantly different (P≤0.05). FIG. 1C is a vertical bar graph showing severity of bacterial speck on tomato plants treated with spray of 4-12 mM solution phenylalanine. Disease was evaluated on a 0-100% severity of symptoms coverage and calculation of area under disease progress curve (AUDPC) during 14 days after treatment. Columns followed by a different letter are significantly different (P<0.05).

FIG. 2 is a bar graph showing severity of symptoms of tomato brown rugose fruit virus (TBRFV) on tomato plants treated with drench or spray of 4 mM solution phenylalanine. Disease was evaluated on a 0-100% severity of symptoms coverage and calculation of area under disease progress curve (AUDPC) during 13 days after treatment. Bars=Standard Errors; Columns followed by a different letter are significantly different (P≤0.05).

FIGS. 3A-3E are images and bar graphs showing the effect of 4 mM of phenylalanine applied by drenching or spraying on the severity of bacterial speck in tomato, 13 days post treatment. FIG. 3A—an image showing the damage caused by the tomato leaf miner, the insect moth Tuta absoluta on tomato leaflet. FIG. 3B—a vertical bar graph showing severity of damage of Tuta absoluta on tomato leaves treated with drench or spray of 4 mM solution phenylalanine. Disease was evaluated on a 0-100% severity of symptoms coverage and calculation of area under disease progress curve (AUDPC) 13 days after treatment. Bars=Standard Errors; Columns followed by a different letter are significantly different (P≤0.05). Symptoms incidence per plant was evaluated 30 days after treatment. Bars=Standard Errors; Columns followed by a different letter are significantly different (P≤0.05). FIG. 3C—a vertical bar graph showing the effect of 4 mM Phe solution on the incidence of tomato leaf miner on tomato plants. The Phe solution was applied by spraying and by drench on the tomato plants at 3 days before and 4 hours before incubation in the greenhouse. Infection was natural. FIG. 3D—a vertical bar graph showing the effect of 4 mM Phe solution on the incidence of tomato leaf miner on tomato plants. The Phe solution was applied by spraying and by drench on the tomato plants at 3 days before and 4 hours before incubation in the greenhouse. Infection was natural. Symptoms incidences per plant at large size and at smaller size (up to 1 cm) were evaluated 30 days after treatment. Bars=Standard Errors; Columns in each symptom size followed by a different letter are significantly different (P≤0.05). FIG. 3E—a vertical bar graph showing the effect of 4 mM Phe solution on the rate of tomato leaf miner symptoms on tomato plants. The Phe solution was applied by spraying and by drench on the tomato plants at 3 days before and 4 hours before incubation in the greenhouse. Infection was natural. Symptoms incidences per plant at large size and at smaller size (up to 1 cm) were evaluated 30 days after treatment and the percent of large symptoms was calculated. Bars=Standard Errors; Columns followed by a different letter are significantly different (P≤0.05).

FIGS. 4A-4B are graphs showing induced resistance of harvested Mango fruit to fungal pathogens. FIG. 4A Mango fruit of the cultivar ‘Shelly’ were dipped for 30 seconds in 4 mM phenylalanine. A day later the treated or untreated fruits were wound inoculated on four spots in each fruit with 7 μl of Lasiodiplodia theobromas conidia suspension (10⁶ conidia/ml) and the decay area was monitored for 7 days post inoculation (dpi). FIG. 4B Mango fruit of the cultivar ‘Shelly’ were dipped for 30 seconds in 4 mM phenylalanine. A day later the treated or untreated fruits were wound inoculated on four spots in each fruit with 7 μl of Colletotrichum gloeosporioides conidia suspension (10⁶ conidia/ml) and the decay area was monitored for 14 days post inoculation (dpi).

FIGS. 5A-5B are a graph and a photograph showing induced resistance of harvested Avocado fruit to fungal pathogens. FIG. 5A is a graph showing the decay area of Avocado fruit of the cultivar ‘Ettinger’ which were dipped for 30 seconds in 1 mM or 4 mM phenylalanine. A day after dipping the treated or untreated fruits were wound inoculated on four spots in each fruit with 7 μl of Alternaria alternata conidia suspension (10⁶ conidia/ml) and the decay area was monitored for 6 days post inoculation (dpi). FIG. 5B is representative pictures of inoculated fruits of control and treatments with 4 mM phenylalanine, 6 days post inoculation.

FIGS. 6A-6C are graphs and a photograph showing induced resistance of harvested clementine fruit to fungal pathogens. FIG. 6A is a vertical bar showing the % of decayed Clementine fruit (in a box) of the cultivar ‘Michal’ which were dipped for 30 seconds in 4 mM phenylalanine. After the fruits were dried, they were sprayed with conidia suspension of Penicillium digitatum (early infection), or alternatively the fruit was sprayed with conidia suspension two days after being dipped in phenylalanine (late infection). FIG. 6B is representative pictures of inoculated fruits of control and treatment with 4 mM phenylalanine, 5 days post inoculation. FIG. 6C is diagrams of taste evaluation of control fruit (upper panel) and phenylalanine treated fruit (lower panel) which include off-flavors (index 1-5), acidity (index 1-5), sweetness (index 1-5), and general taste (index 1-5).

FIGS. 7A-7B are a graph (7A) and a photograph (7B) showing that the protective effect of sprayed Phe. Resistance to Botrytis was apparent at 6 mM or more Phe. Interestingly, up to 35 mM, no sedimentation of the sprayed Phe was noticed.

FIGS. 8A-8B are a graph (8A) and a photograph (8B) showing that the protective effect of drenching Phe. Resistance to Botrytis was apparent at 6 mM or more Phe. Interestingly, up to 35 mM, no sedimentation of the drenched Phe was noticed.

FIG. 9 is a bar graph showing the effect of 4 mM Phe solution on the severity of Tetranychus urticae red spider mite on leaves of tomato plants. The Phe solution was applied by spraying and by drench on the tomato plants at 3 days before and 4 hours before incubation in the greenhouse. Symptoms severity was evaluated on a scale of 0-100% severity of symptoms coverage during 30 days after treatment and it is expressed as the area under disease progress curve (AUDPC). Bars=Standard Errors; Columns followed by a different letter are significantly different (P≤0.05).

FIG. 10 is a bar graph showing the effect of 4 mM Phe solution on the severity of silver leaf white fly (SLWF) on tomato plants. The Phe solution was applied by spraying and by drench on the tomato plants at 3 days before and 4 hours before incubation in the greenhouse. Incidence of SLWF was evaluated on a representative leaf 30 days after treatment. Bars=Standard Errors; Columns followed by a different letter are significantly different (P≤0.05).

FIGS. 11A-11B are bar graphs showing: FIG. 11A: the effect of 4 mM Phe solution on the severity of Oidium neolycopersici powdery mildew on tomato plants. The Phe (P) solution was applied by spraying and by drench on the tomato plants at 3 days before and 4 hours before incubation in the greenhouse. Incidence of Oidium powdery mildew was evaluated 24 days after treatment. Bars=Standard Errors; Columns followed by a different letter are significantly different (P≤0.05). FIG. 11B: the effect of 4 mM Phe solution and 0.1% Alkyl Phenol Ethylene Oxide (Shatah 90, Adama Makhteshim) on the severity of Oidium neolycopersici powdery mildew on tomato plants. The Phe and shatakh 90 solutions were applied by spraying on the tomato plants at 3 days before and 4 hours before incubation in the greenhouse. Incidence of Oidium powdery mildew was evaluated 24 days after treatment. Bars=Standard Errors; Columns followed by a different letter are significantly different (P≤0.05).

FIG. 12 is a bar graph showing the effect of 4 mM Phe solution on the severity of Oidiopsis sicula powdery mildew on tomato plants. The Phe solution was applied by spraying and by drench on the tomato plants at 3 days before and 4 hours before incubation in the greenhouse Incidence of Oidiopsis powdery mildew was evaluated 24 days after treatment. Bars=Standard Errors; Columns followed by a different letter are significantly different (P≤0.05).

FIG. 13 is a bar graph showing the effect of 4 mM solution on the severity of Tomato yellow leaf curl virus (TYLCV) on leaves of tomato plants. The Phe solution was applied by spraying and by drench on the tomato plants at 3 days before and 4 hours before incubation in the greenhouse close to TYLCV infected tomato plants. Infection was natural by the vector insect silverleaf white fly. Disease severity was evaluated on a scale of 0-100% severity of symptoms coverage during 51 days after treatment and it is expressed as the area under disease progress curve (AUDPC). Bars=Standard Errors; Columns followed by a different letter are significantly different (P≤0.05).

FIG. 14 is a bar graph showing the effect of 2-4 mM Phe solution on the severity of tomato gray mold on leaves of tomato plants. The Phe solution was applied by drench to the root zone of the tomato plants at 3 days before and 4 hours before incubation in high humidity in a growth room with temperature of 19-20° C. As shown, 4 mM Phe resulted in significantly better disease control effect than 2 mM.

FIG. 15 is a bar graph showing the effect of 2-8 mM solution on the severity of tomato gray mold on leaves of tomato plants. The Phe solution was sprayed the tomato plants at 3 days before and 4 hours before incubation in high humidity in a growth room with temperature of 19-20° C. As shown, 4 and 8 mM Phe resulted in significantly better disease control effect than 2 mM.

FIGS. 16A-16B are bar graphs showing the effect of 1-4 mM solution on the severity of tomato gray mold on leaves of tomato plants. The Phe solution was sprayed the tomato plants (16A) or drenched to the tomato plants root zone (16B) at 3 days before and 4 hours before incubation in high humidity in a growth room with temperature of 19-20° C. Infection was made by wounding the leaf tissue and placing a drop of Botrytis cinerea suspension on the wound. As shown, 4 mM Phe resulted in significantly better disease control effect than 2 mM.

FIGS. 17A-17B is bar graphs showing the effect of 2-4 mM solution on the severity of tomato gray mold on leaves of tomato plants. The Phe solution was sprayed the tomato plants (17A) or drenched to the tomato plants root zone (17B) at 3 days before and 4 hours before incubation in high humidity in a growth room with temperature of 19-20° C. Infection was made by wounding the leaf tissue and placing a drop of Botrytis cinerea suspension on the wound. As shown, 4 mM Phe resulted in significantly better disease control effect than 2 mM.

FIG. 18 is a graph showing the effect of Phe solution on fruit pre-harvest decay. The graph shows the decay incidence of control strawberry fruits or treated fruits (sprayed 3-weeks pre-harvest). Decay was monitored and documented over a period of 12 days.

FIGS. 19A-19G are images and graphs showing the effect of Phe solution on fruit post-harvest rotting under cold temperature resistance. (19A-19B) are images showing post-harvest control and treated mango fruits, respectively, stored in 7° C. Control fruits were shown to have substantially more black spots, indicating infestation of pesticides. (19C-19D) are vertical bar graphs showing indices of spots (19C) and pitting (19D) of mango fruits infected by Colletotrichum or Lasiodiplodia under 7° C. (19E-19G) are vertical bar graph showing serum decay % (19E), side decay % (19F), and total decay % (19G) of fruits. Serum decay was shown at 7° C., and side decay and total decay were shown at 7° C. and 10° C.

FIGS. 20A-20I are images and graphs showing the effects of Phe solution on plant growth performance parameters. (20A) is images of plants treated with 2 mM once or twice, once with 4 mM or non-treated (control). (20B-20C) are bar graphs showing plant height (20B) and the % increase in the plant height (20C) in treated and control plants. (20D-20E) are bar graphs showing the number of nodes per plant (20D) and the % increase in the number of nodes (20E) in treated and control plants. (20F-20G) are bar graphs showing the % increase in leaf length (20F) and the % increase in the leaf area (20G) in treated and control plants. (20H-20I) are bar graphs showing plant fresh (e.g., “wet”) weight (20G) and plant dry weight (20I) in treated and control plants.

FIGS. 21A-21D are bar graphs showing the effects of 1-4 mM solution on plant crop production (e.g., fruits). Plants were treated with 2 mM once or twice, once with 4 mM or non-treated (control). (21A) is a graph showing the cumulative weight of fruits per plant. (21B) is a graph showing the number of fruits per plant. (21C) is a graph showing the average weight of a fruit. (21D) is a graph showing the number of young fruits, e.g., “set fruit”.

FIGS. 22A-22C are vertical bar graphs showing the effect of Phe solution on the number of flowers per plant. Petunia plants were treated once a week for 5 weeks with Phe solution (2 mM, 6 mM, 15 mM, 35 mM, or none, i.e., control). Numbers of flowers were counted and documented over a period of 7 weeks for Petunia ‘easy-rider’ strain (FIG. 22A) and Petunia ‘white dreams’ strain (FIG. 22B). Plants treated with Phe solution, were also shown to have more stigmas compared to control (FIG. 22C).

FIGS. 23A-C: Effect of flowering application on disease incidence. Avocado and Mango cv. ‘Kent’ orchards were sprayed with fungicide (Pyrimethanil+Fludioxonil) or with phenylalanine during flowering. (FIG. 23A). Powdery Mildew incidence (percentage) per inflorescence in mango orchard. (FIG. 23B). Anthracnose incidence (percentage) on harvested mango stored at ambient temperature for 12 days. (FIG. 23CB). Anthracnose incidence (percentage) on harvested avocado stored at ambient temperature for 14 days.

### DETAILED DESCRIPTION OF THE INVENTION

The present invention, in some embodiments thereof, relates to a method of controlling pathogen infections and arthropod or pathogen damages in plants. The present invention, in some embodiments thereof, relates to a method of improving the performance of a plant. In some embodiments, improved performance of a plant is directed to one or more outcomes selected from: growth, crop and resistance, e.g., to abiotic stressors.

A plant, according to some embodiments, is any part or tissue of a plant. A plant, according to some embodiments, is a fruit. A plant, according to some embodiments, is a root. A plant, according to some embodiments, is a leaf. A plant, according to some embodiments, is a seed. A plant, according to some embodiments, is a harvested plant.

In one embodiment, a pathogen is any biotic factor, pest or organism that damages and/or infect a plant. In one embodiment, a pathogen is any biotic factor, pest or organism that alters the plant's appearance. In one embodiment, a pathogen is any biotic factor, pest or organism that alters the plant's biochemical content. In one embodiment, a pathogen is any biotic factor that resides on or within the plant.

As used herein, the term “aromatic amino acid (AAA)”, refers to phenylalanine (Phe), Tyrosine (Tyr), Tryptophan (Trp), or any analog thereof. As used herein, any AAA analog is applicable as long as the analog maintains the anti-pathogenic and performance improving activities, as disclosed herein.

In some embodiments, an AAA, for example Phe, as utilized herein protects plants from the destructive effects of a pathogen such as but not limited to a bacterium, a virus, a moth, an insect or any combination thereof. In some embodiments, AAA inhibits, eliminates, reduces the risk of a pathogen. In some embodiments, AAA protects a plant against pathogenic infection. In some embodiments, AAA treats a plant afflicted with a pathogen. In some embodiments, an activity of AAA as described herein is in a dose dependent manner.

It is therefore contemplated that AAA, such as Phe or analogs thereof, can be safely used as an agent against pest and pathogenic stress and/or damage.

Thus, according to an aspect, there is provided a method of controlling a pathogenic infection in a plant, comprising applying to the plant or contacting the plant with an effective amount of a composition comprising an AAA.

As used herein, a composition of the invention comprises AAA for use in controlling a pathogenic infection in a plant. In some embodiments, the composition of the invention comprises AAA for use in improving performance of a plat, wherein improved performance comprises one or more outcomes selected from: increased growth, increased crop yields, and increased resistance.

In some embodiments, the composition of the invention comprises Phe or an analog thereof. In some embodiments, the composition of the invention comprises Tyr or an analog thereof. In some embodiments, the composition of the invention comprises Trp or an analog thereof. In some embodiments, the composition of the invention comprises Phe or an analog thereof, and Tyr or an analog thereof In some embodiments, the composition of the invention comprises Phe or an analog thereof, and Trp or an analog thereof. In some embodiments, the composition of the invention comprises Tyr or an analog thereof, and Trp or an analog thereof. In some embodiments, the composition of the invention comprises Phe or an analog thereof, Tyr or an analog thereof, and Trp or an analog thereof.

In one embodiment, a composition comprising AAA is used for inhibiting, ameliorating, treating, preventing and/or controlling a pathogenic infection in the plant, wherein a concentration of AAA, such as phenylalanine or an analog thereof, is 2 mM, 3 mM, 4 mM, 8 mM or above 2 mM. In one embodiment, a concentration of AAA, such as phenylalanine, above 2 mM is used for inhibiting, ameliorating, treating, preventing and/or controlling a pathogenic infection wherein the pathogenic infection is not a fungal infection. In one embodiment, preventing and/or controlling a pathogenic infection includes the elimination of a plant pathogen. In one embodiment, preventing and/or controlling a pathogenic infection includes inhibiting the activity and/or the pathogenic potential of a plant pathogen. In one embodiment, preventing and/or controlling a pathogenic infection includes inhibiting the spread of the pathogen within a plant or to a neighboring plant.

According to an alternative or an additional aspect there is provided a method of controlling a viral infection in a plant, the method comprising applying to the plant an effective amount of an AAA, such as phenylalanine or an analog thereof, or a composition comprising thereof, for controlling the viral infection in the plant.

According to an embodiment, provided herein is a method of controlling a pathogenic infection in a plant susceptible thereto such as but not limited to a neighboring plant (a plant in proximity of up to 200 km to an infected plant), the method comprising applying to the plant an effective amount of an AAA, such as phenylalanine or an analog thereof, or a composition comprising thereof, for controlling or reducing the risk of pathogenic infection in the plant. In one embodiment, the pathogen is a bacterium. In one embodiment, the pathogen is a Pseudomonas. In one embodiment, the pathogen is a plant virus. In one embodiment, the plant virus is tomato brown rugose fruit virus (TBRFV). In one embodiment, the plant virus is Cucurbit yellow stunting disorder virus (CYSDV). In one embodiment, the plant virus is Cucurbit chlorotic yellows virus (CCYV). In one embodiment, the pathogen is Pseudomonas syringae pv tomato. In one embodiment, the pest is Tuta absoluta.

As used herein the term “controlling” refers to preventing, inhibiting, ameliorating a symptom or reducing pathogen infection or arthropod damage or inhibiting the rate, spread and extent of such infection. Curative treatment is also contemplated herein. In one embodiment, controlling comprises inhibiting or stopping the spread of an infection from one plant to another plant. In one embodiment, another plant is a neighboring plant. In one embodiment, inhibiting or stopping the spread of an infection from one plant to another plant comprises treating the infected plant, a neighboring plant or both with AAA, such as Phe or an analog thereof, or with a composition comprising thereof, as described herein.

In some embodiments, controlling is treating a plant afflicted with a disease caused by a biotic stressor. In some embodiments, controlling is reducing the risk of infection of uninfected plant or reducing the risk of infection in an uninfected portion of a plant that is infected in other portions. In some embodiments, controlling is reducing the risk of an infection by a stressor as described herein. In some embodiments, controlling is inhibiting or eliminating a plant biotic stressor. In some embodiments, controlling is reducing the risk of contact between a plant and a biotic stressor. In some embodiments, controlling is treating a plant disease caused by a biotic stressor. In some embodiments, a stressor comprises a pathogen. In some embodiments, controlling is ameliorating a pathology associated with infection or resulting from infection as described herein.

According to an embodiment, the biotic stressor is a virus. According to an embodiment, the virus is of the Virgaviridae family. According to an embodiment the virus is a tomato brown rugose fruit virus (TBRFV). According to an embodiment, the virus is a tobamovirus. According to an embodiment, the virus is Cucurbit yellow stunting disorder virus (CYSDV). According to an embodiment, the virus is Cucurbit chlorotic yellows virus (CCYV).

According to an embodiment, the bacterium is of the family Pseudomonadaceae. According to an embodiment, the bacterium is Pseudomonas syringae.

According to an embodiment, the controlling is of damage caused by an insect. In one embodiment, controlling is minimizing, inhibiting or reversing a damage.

According to an embodiment, the insect is of the family Gelechiidae. According to an embodiment, the insect is a moth such as but not limited to: Tuta absoluta. According to an embodiment, the controlling is of a pathogenic infection by a nematode.

According to an embodiment, the nematode is selected from the groups of Aphelenchoides (foliar nematodes), Ditylenchus, Globodera (potato cyst nematodes), Heterodera (soybean cyst nematodes), Longidorus, Meloidogyne (root-knot nematodes), Nacobbus, Pratylenchus (lesion nematodes), Trichodorus and Xiphinema (dagger nematodes).

Following are further examples of plant pathogens and arthropod pests that are contemplated targets for control according to some embodiments of the invention as well as some diseases caused thereby.

TABLE 1
Diseases caused by bacteria and controlled by the composition and methods
described herein

| Disease name | Causal agent | Crops |
|---|---|---|
| Deep pitted scab, scab | Streptomyces spp. | Peanut, potato, carrot |
| Bacterial soft rot | Pectobacterium | Alfala, Sweet potato, sunflower, |
| | carotovorum subsp. | maize, potato, onion, carrot, |
| | carotovorum | cucurbits, lettuce, eggplant, celery, |
| | | Brassica spp., tomato, faba bean, |
| | | pepper, artichoke, garlic, bean, |
| | | avocado, banana, asclepias, |
| | | gypsophila, anemone, liatris, |
| | | pelargonium, carnation, lily, chard, |
| | | chives |
| Bacterial blight | Pseudomonas syringae | Alfala, Wheat, barley, avocado |
| | pv. syringae | |
| Bacterial brown spot, leaf | Pseudomonas syringae | Pea, cucurbits, pepper |
| spot | pv. syringae | |
| Sweet-potato scab, | Streptomyces scabies | Sweet potato, potato |
| common scab | | |
| Black chaff, bacterial leaf | Xanthomonas | Barley |
| streak | translucens pv. | |
| | translucens | |
| Bacterial leaf spot | Pseudomonas syringae | Clover |
| Bacterial fruit blotch | Acidovorax avena subsp. | Cucurbits |
| | citrulli | |
| Bacterial leaf spot and | Xanthomonas campestris | Lettuce |
| head rot | | |
| Bacterial leaf spots of | Xanthomonas campestris | Carrot, celery |
| Umbelliferae | pv. carotae | |
| Bacterial spot | Xanthomonas campestris | Tomato, pepper |
| | pv. vesicatoria | |
| Bacterial canker | Clavibacter | Tomato, pepper |
| | michiganensis subsp. | |
| | michiganensis | |
| Pith necrosis | Pseudomonas corrugata | Tomato |
| Bacterial speck | Pseudomonas syringae | Tomato |
| | pv. tomato | |
| Stewart's wilt of corn | Erwinia stewartii | Maize |
| Fireblight | Erwinia amylovora | Apple, crabapples, loquat, pear |
| Bacterial crown gall | Rhizobium radiobacter | Sweet potato, rose, grapevine, |
| | | carrot, beet, tomato, pepper, |
| | | avocado, pear, peach, cherry, olive, |
| | | apricot, plum, loquat, almond, fig, |
| | | apple, eucalyptus, aster, anemone, |
| | | buttercup, carnation, chard |
| Citrus canker | Xanthomonas | Citrus |
| | axonopodis | |
| Citrus greening | Candidatus Liberibacter | Citrus |
| Bacterial blight | Xanthomonas oryzae pv. | Rice |
| | oryzae | |
| Bacterial leaf streak | Xanthomonas oryzae pv. | Rice |
| | oryzicola | |
| Foot rot | Erwinia chrysanthemi. | Rice |
| Grain rot | Burkholderia glumae | Rice |

TABLE 2
Diseases caused by viruses

| Disease name | Virus | Crops |
|---|---|---|
| Peanut mottle | Peanut mottle virus (PMV) | Peanut |
| Potyvirus | | |
| Peanut stunt | Peanut stunt virus (PSV) | Peanut |
| Tomato spotted wilt | Tomato spotted wilt virus | Peanut, tomato |
| | (TSWV) Tospovirus | |
| Cowpea mild mottle | Cowpea mild mottle virus | Peanut, tomato |
| | (CMMCv) Carlavirus | |
| Alfalfa mosaic | Alfalfa mosaic virus (AMV) | Alfalfa, chickpea, clover, |
| | Alfamovirus | potato, carrot |
| Cucumber mosaic | Cucumber mosaic virus (CMV) | Sweet potato, chickpea, |
| | Cucumovirus | clover, carrot, cucurbits, |
| | | eggplant, celery, tomato, |
| | | pepper, bean, avocado, |
| | | amaryllis, asclepias, |
| | | delphinium, trachelium, |
| | | anemone, lisianthus, |
| | | buttercup, limonium, phlox, |
| | | lily, spinach, beet |
| Sweet-potato virus C | Sweet-potato virus C (SPVC) | Sweet potato |
| | potyvirus | |
| Barley yellow dwarf | Barley yellow dwarf virus | Barley, wheat |
| | (BYDV) Luteovirus | |
| Narrow leaf | Bean yellow mosaic virus | chickpea |
| | (BYMV) Begomovirus | |
| Maize dwarf mosaic | Maize dwarf mosaic virus | Maize, Sorghum |
| | (MDMV) Potyvirus | |
| Maize rough dwarf | Maize rough dwarf virus | Maize |
| | (MRDV) Fijivirus | |
| Potato leafroll | Potato leafroll virus (PLRV) | Potato, tomato |
| | Luteovirus | |
| Tobacco mosaic | Tobacco mosaic virus (TMV) | Potato, tomato, lisianthus, |
| | Tobamovirus | tobacco, pepper, potato, |
| | | eggplant, cucumber and |
| | | petunia |
| Potato virus X | Potato virus X (PVX) Potexvirus | Potato |
| Potato rugose mosaic, | Potato virus Y (PVY, strains O, N | Potato, Eggplant, tomato |
| virus Y | and C) Potyvirus | |
| Zucchini yellow fleck | Zucchini yellow fleck virus | Cucurbits |
| | (ZYFV) Potyvirus | |
| Watermelon chlorotic | Watermelon chlorotic stunt virus | Cucurbits |
| stunt | (WmCSV) Begomoviruses | |
| Cucurbit yellow | Cucurbit yellow stunting disorder | Cucurbits |
| stunting disorder | virus (CYSDV) Crinivirus | |
| Cucumber vein | Cucumber vein yellowing virus | Cucurbits |
| yellowing | (CVYV) Ipomovirus | |
| Squash leaf curl | Squash vein yellowing virus | Cucurbits |
| | (SqVYV) Ipomovirus | |
| Papaya ring spot | Papaya ring spot virus W strain | Cucurbits |
| | (PRSV-W) Potyvirus | |
| Papaya mosaic | Papaya mosaic virus (PapMV) | Cucurbits |
| | Potyvirus | |
| Zucchini yellows | Zucchini yellows mosaic virus | Cucurbits |
| mosaic | (ZYMV) Potyvirus | |
| Cucumber fruit mottle | Cucumber fruit mottle mosaic | Cucurbits |
| mosaic | virus (CFMMV) Tobamovirus | |
| Cucumber leaf spot | Cucumber leaf spot virus (CLSV) | Cucurbits |
| | Tombusvirus | |
| Squash leaf curl | Squash leaf curl virus (SqLCV) | Cucurbits |
| | Begomovirus | |
| Lettuce mosaic | Lettuce mosaic virus (LMV) | Lettuce |
| | Potyvirus | |
| Lettuce big vein | Lettuce big-vein associated virus | Lettuce |
| | (LBVaV) Varicosavirus; | |
| | Mirafiori lettuce virus | |
| | (MiLV) Ophiovirus | |
| Beet western yellows | Beet western yellows virus | Lettuce |
| | (BMYV) Polerovirus | |
| Tomato spotted wilt | Tomato spotted wilt virus | Eggplant, pepper, tomato |
| | (TSWV) Tospovirus | |
| Eggplant mottled | Eggplant mottled crinkle mosaic | Eggplant |
| crinkle mosaic | virus (EMCV) | |
| Eggplant mild mottled | Eggplant mild mottle virus | Eggplant |
| | (EMMV) | |
| Tomato mild mottle | Tomato mild mottle virus | Eggplant |
| | (TMMV) Tombusvirus | |
| Tomato mosaic | Tomato mosaic virus (ToMV) | Tomato, pepper |
| | Tobamovirus | |
| Tomato chlorosis | Tomato chlorosis virus (TOCV) | Tomato |
| | Crinivirus | |
| Pelargonium zonate | Pelargonium zonate spot virus | Tomato, pelargonium |
| spot | (PZSV) Anulavirus | |
| Tomato yellow leaf curl | Tomato yellow leaf curl virus | Tomato, bean |
| | (TYLCV) Begomovirus | |
| Pepper mild mottle | Pepper mild mottle virus | Pepper |
| | (PMMoV) Tobamovirus | |
| Pepper yellow leaf curl | Pepper yellow leaf curl virus | Pepper |
| | (PYLCV | |
| Iris yellow spot | Iris yellow spotted virus (IYSV) | Onion, Iris |
| | Tospovirus | |
| Onion yellow dwarf | Onion yellow dwarf virus | Onion, garlic |
| | (OYDV) Potyvirus | |
| Bean common mosaic | Bean common mosaic (BCMV) | Bean |
| | Potyvirus | |
| Bud blight | Soybean bud blight virus (SBBV) | soybean |
| Sugarcane mosaic | Sugarcane mosaic virus (SMV) | Sugarcane |
| Cauliflower mosaic | Cauliflower mosaic virus (CMV) | Cabbage, Brussels sprouts, |
| | | cauliflower, broccoli and |
| | | rape seed |
| African cassava mosaic | African cassava mosaic virus | Cassava |
| | (ACMV) | |
| Plum pox | Plum pox virus (PPV) | Plum |
| Tristeza | Citrus tristeza virus (CTV) | Citrus |
| Tomato bushy stunt | Tomato bushy stunt virus (TBSV) | Tomato |
| Rice tungro spherical | Rice tungro spherical virus | Rice |
| | (RTSV) | |
| Rice yellow mottle | Rice yellow mottle virus (RYMV) | Rice |
| Rice hoja blanca | Rice hoja blanca virus (RHBV) | Rice |
| Maize streak | Maize streak mastrevirus (MSV) | Maize |
| Maize rayado fino | Maize rayado fino virus (MRFV) | Maize |
| Sweet potato feathery | Sweet potato feathery mottle | Sweet potato |
| mottle | potyvirus (SPFMV) | |
| Sweet potato sunken | Sweet potato sunken vein | Sweet potato |
| vein | closterovirus (SPSVV) | |
| African cassava mosaic | African cassava mosaic disease | Cassava |
| disease | begomovirus complex (ACMD) | |
| | complex of ACMV, EACMV, | |
| | SACMV | |
| Banana bunchy top | Banana bunchy top nanovirus | Banana |
| | (BBTV) | |
| Banana streak | Banana streak badnavirus (BSV) | Banana |

TABLE 3
Insect pests

| Common name | Scientific species | Crops |
|---|---|---|
| Tomato leafminer | Tuta absoluta | Tomato, |
| | | potato, eggplant, pepino, |
| | | pepper, tobacco |
| Olive fly | Bactrocera oleae | Olive |
| Greater date | Aphomia (Arenipses) sabella | Date palm |
| moth | | |
| Sweet potato | Bemisia tabaci | Tomato, peppers, squash, |
| whitefly | | cucumber, beans, eggplant, |
| | | watermelon, cabbage, potato, |
| | | peanut, soybean, cotton many |
| | | ornamental host plants: |
| | | poinsettia, hibiscus, |
| | | chrysanthemum and any other |
| | | crops |
| Corn aphid | Rhopalosiphum maidis | Sorghum, cereals |
| Cowpea aphid | Aphis craccivora | Bean, cowpea |
| Green peach | Myzus persicae | Peach, cotton, rapeseed |
| aphid | | |
| Oat aphid | Rhopalosiphum padi | Barley, wheat, oats |
| Pea aphid | Acyrthosiphon pisum | Chickpea, pea, lucerne |
| Rice root aphid | Rhopalosiphum rufiabdominalis | Rice |
| Soybean aphid | Aphis glycine | Soybean, Glycine spp. |
| Spotted alfalfa | Therioaphis trifolii | Lucerne |
| aphid | | |
| Turnip aphid | Lipaphis erysimi | Rapeseed |
| Wheat aphid | Rhopalosiphum padi | Barley, wheat and oats |
| Common | Leucania convecta | barley, oats, wheat, native |
| armyworm | | pasture grasses and perennial |
| | | grass seed crops |
| Northern | Leucania separata | sorghum, maize, barley, wheat |
| armyworm | | and rice |
| Large brown | Riptortus serripes | Soybeans, pulses, cotton and |
| bean bug | | many horticultural crops |
| Small brown | Melanacanthus scutellaris | Pulses |
| bean bug | | |
| Brown flea | Chaetocnema sp. | Cotton |
| beetle | | |
| Brown mind | Creontiades pacificus | Cotton, lucerne, mungbeans, |
| | | navy beans, peanut and soybeans |
| Cabbage moth | Plutella xylostella | Brassica plants |
| Budworm | Helicoverpa punctigera | cotton, chickpea, sunflower, |
| | | soybean, mungbean, navy bean, |
| | | lucerne, canola, peanut, faba |
| | | bean, safflower, linseed |
| Cotton bollworm, | Helicoverpa armigera | , wheat and barley |
| corn earworm | | |
| Crop mirid | Sidnia kinbergi | Cotton, lucerne, mungbeans, |
| | | navy beans, peanut, soybeans |
| Diamondback | Plutella xylostella | Rapeseed |
| moth | | |
| False wireworm | Pterohelaeus and | Maize |
| | Gonocephalum spp | |
| Greenhouse | Trialeurodes vaporariorum | Cotton, sunflower, soybean and |
| whitefly | | navy bean |
| Cotton | Amrasca terraereginae | Cotton |
| Leafhopper | | |
| Lesser | Spodoptera exigua | Cotton |
| armyworm | | |
| Onion thrips | Thrips tabaci | Cotton, navy bean, mungbean, |
| | | cereals |
| Soybean | Porphyrosela aglaozona | Soybean |
| leafminer | | |
| Soybean looper | Thysanoplusia orichalcea | Soybean |
| Soybean moth | Aproaerema simplexella | Soybean |
| Tobacco Thrips | Thrips tabaci | Cotton, bean, mungbean, cereals |
| Western flower | Frankliniella orientalis | Cotton, navy bean, mungbean, |
| thrips | | sunflower, canola, peanut, other |
| | | crops |

TABLE 4
Arachnid pests

| Common name | Scientific species | Crops |
|---|---|---|
| Broad mite | Polyphagotarsonemus | Pepper, sweet basil, apple, |
| | latus | avocado, cantaloupe, castor, |
| | | chili, citrus, coffee, cotton, |
| | | eggplant, grapes, guava, jute, |
| | | mango, papaya, passion fruit, |
| | | pear, potato, sesame, beans, |
| | | tea, tomato, African violet, |
| | | ageratum, azalea, begonia, |
| | | chrysanthemum, cyclamen, |
| | | dahlia, gerbera, gloxinia, ivy, |
| | | jasmine, impatiens, lantana, |
| | | marigold, peperomia, |
| | | pittosporum, snapdragon, |
| | | verbena, zinni |
| Mite, Bean spider | Tetranychus | Cotton, bean, soybean |
| | ludeni | |
| Two-spotted Mite | Tetranychus | horticultural and field crops, |
| | urticae | including maize, cotton, |
| | | soybean, canola, lucerne, |
| | | peanut, mungbean, beans |

### Improved Performance

According to some embodiments, there is provided a method of improving the performance of a plant, comprising contacting the plant with an effective amount of an aromatic amino acid (AAA), such as phenylalanine, or an analog thereof, or any combination thereof, or a composition comprising thereof, thereby improving the performance of a plant.

As used herein, improved plant performance comprises one or more outcomes selected from: increased plant growth, increased crop yields, and increased resistance.

As used herein, increased growth comprises one or more parameters selected from: growth rate, plant weight, plant height, leaf length, leaflet area, number of nodes, distance between adjacent nodes, and is relative to a control.

### Growth Parameters

In some embodiments, a plant contacted by an AAA or by a composition comprising thereof grows faster than a control plant. In some embodiments, contacting a plant with an AAA or by a composition comprising thereof increases the plant growth rate by at least 10%, by at least 20%, by at least 50%, by at least 100%, by at least 200%, by at least 350%, by at least 500%, by least 750%, or by at least 1,000% compared to control, and any value and range therebetween. In some embodiments, contacting a plant with an AAA or by a composition comprising thereof increases the plant growth rate by 10-50%, 25-75%, 50-150%, 100-250%, 200-400%, 350-500%, 450-750%, or 700-1,000% compared to control. Each possibility represents a separate embodiment of the invention.

In some embodiments, a plant contacted by an AAA, or by a composition comprising thereof weighs more than a control plant. In some embodiments, contacting a plant with an AAA, or by a composition comprising thereof increases the plant weight by at least 10%, by at least 15%, by at least 25%, by at least 35%, by at least 45%, by at least 55%, by at least 65%, or by at least 80% compared to control, and any value and range therebetween. In some embodiments, contacting a plant with an AAA at a concentration of at least 2 mM, or by a composition comprising thereof increases the weight of the plant by 5-10%, 7-20%, 15-30%, 25-40%, 35-55%, 45-65%, or 50-70% compared to control. Each possibility represents a separate embodiment of the invention.

In some embodiments, the term “weight” refers to dry weight. In other embodiments, weight refers to wet weight.

In some embodiments, a plant contacted by an AAA, or by a composition comprising thereof is higher than a control plant. In some embodiments, contacting a plant with an AAA, or by a composition comprising thereof increases the height of the plant by at least 10%, by at least 15%, by at least 25%, by at least 35%, by at least 45%, by at least 55%, or by at least 65% compared to control, and any value and range therebetween. In some embodiments, contacting a plant with an AAA at a concentration of at least 2 mM, or by a composition comprising thereof increases the height of the plant by 5-10%, 7-20%, 15-30%, 25-40%, 35-55%, or 45-65% higher than control. Each possibility represents a separate embodiment of the invention.

In some embodiments, a plant contacted by an AAA, or by a composition comprising thereof comprises more nodes on its stem compared to a control plant. In some embodiments, contacting a plant with an AAA, or by a composition comprising thereof increases the number of nodes on the plant stem by at least 5%, by at least 10%, by at least 15%, by at least 25%, by at least 35%, by at least 40%, or by at least 45% compared to control, and any value and range therebetween. In some embodiments, contacting a plant with an AAA, or by a composition comprising thereof increases the number of nodes on the plant stem by 5-10%, 7-20%, 15-30%, 25-40%, or 35-50% compared to control. Each possibility represents a separate embodiment of the invention. As a non-limiting example, the number of nodes is determined per length (e.g., per 20 centimeters) in a plant contacted by the composition of the invention and thereafter is compared to the same output recorded in a control plant.

In some embodiments, adjacent nodes on the stem of a plant contacted by an AAA, or by a composition comprising thereof are at a greater distance from one another compared to adjacent nodes on the stem of a control plant. In some embodiments, the distance between a pair of adjacent nodes on the stem of a plant contacted by an AAA, or by a composition comprising thereof is at least 5%, at least 10%, at least 15%, at least 25%, at least 35%, at least 40%, by at least 45%, at least 50%, at least 75%, at least 100%, at least 250%, or at least 500% greater than the distance in a control, and any value and range therebetween. In some embodiments, the distance between a pair of adjacent nodes on the stem of a plant contacted by an AAA, or by a composition comprising thereof is 5-15%, 10-25%, 20-35%, 25-40%, 30-45%, 30-50%, 40-75%, 70-100%, 90-250%, or 200-500% greater than the distance in a control. Each possibility represents a separate embodiment of the invention.

In some embodiments, a plant contacted by an AAA, or by a composition comprising thereof has longer leaves compared to a control plant. In some embodiments, contacting a plant with an AAA, or by a composition comprising thereof increases the length of a leaf of the plant by at least 10%, by at least 20%, by at least 50%, by at least 100%, by at least 200%, by at least 350%, by at least 500%, by least 750%, or by at least 1,000% compared to control, and any value and range therebetween. In some embodiments, contacting a plant with an AAA, or by a composition comprising thereof increases the length of a leaf of the plant by 10-50%, 25-75%, 50-150%, 100-250%, 200-400%, 350-500%, 450-750%, or 700-1,000% compared to control. Each possibility represents a separate embodiment of the invention.

In some embodiments, a leaflet of a plant contacted by an AAA, or by a composition comprising thereof has a bigger surface area compared to a control plant. In some embodiments, contacting a plant with an AAA, or by a composition comprising thereof increases the surface area of a leaflet of the plant by at least 10%, by at least 20%, by at least 50%, by at least 100%, by at least 200%, by at least 350%, by at least 500%, by least 750%, or by at least 1,000% compared to control, and any value and range therebetween. In some embodiments, contacting a plant with an AAA, or by a composition comprising thereof increases the surface area of a leaflet of the plant by 10-50%, 25-75%, 50-150%, 100-250%, 200-400%, 350-500%, 450-750%, or 700-1,000% compared to control. Each possibility represents a separate embodiment of the invention.

As used herein, a control refers to plant which is or was not contacted by an AAA, or a composition comprising thereof. As used herein, the terms “increase”, “increased”, “increasing” are relative to a control plant, as described hereinabove.

As used herein, increased crop comprises one or more parameters selected from: increased number of young fruits (e.g., “set fruits”), increased fruit yield, or a combination thereof.

In some embodiments, a plant contacted by an AAA, or by a composition comprising thereof has more young fruits e.g., set fruit, compared to a control plant. In some embodiments, a plant contacted by an AAA, or by a composition comprising thereof sets more young fruits compared to a control plant.

Identification and determination of the presence and quantity of young fruits are common and would be apparent to one of ordinary skill in the art.

In one embodiment, the number of young fruits reflects the yield potential of a plant. In one embodiment, the number of young fruits positively correlates with the yield of a plant. In one embodiment, the number of young fruits is a predictive indicator of the yield potential of a plant. In some embodiments, at least 30%, at least 40%, at least 50%, at least 75%, at least 85%, at least 90%, or at least 95% of young fruits develop or mature into full fruits or consumable fruits, and any value and range therebetween. In some embodiments, 25-40%, 35-50%, 45-75%, 60-85%, 70-90%, or 85-99% of young fruits develop or mature into full fruits or consumable fruits. Each possibility represents a separate embodiment of the invention.

In some embodiments, contacting a plant with an AAA, or by a composition comprising thereof increases the number of young fruits (e.g., set fruit) of the plant by at least 10%, by at least 20%, by at least 50%, by at least 100%, or by at least 200%, compared to control, and any value and range therebetween. In some embodiments, contacting a plant with an AAA, or by a composition comprising thereof increases the number of young fruits (e.g., set fruit) of the plant by 10-50%, 25-75%, 50-150%, 100-250%, 200-400%, 350-500%, 450-750%, or 700-1,000% compared to control. Each possibility represents a separate embodiment of the invention.

In some embodiments, a plant contacted by an AAA, or by a composition comprising thereof has more fruits, compared to a control plant. As used herein, “more fruits” is measured by cumulative fruit weight, by fruit number, by fruit weight per plant, by fruit weight per plant weight, by fruit number per plant weight, or any combination thereof.

In some embodiments, contacting a plant with an AAA, or by a composition comprising thereof increases the number of fruits of the plant by at least 10%, by at least 20%, by at least 50%, by at least 100%, or by at least 200%, compared to control, and any value and range therebetween. In some embodiments, contacting a plant with an AAA, or by a composition comprising thereof increases the number of fruits of the plant by 10-50%, 25-75%, 50-150%, 100-250%, 200-400%, 350-500%, 450-750%, or 700-1,000% compared to control. Each possibility represents a separate embodiment of the invention.

In some embodiments, contacting a plant with an AAA, or by a composition comprising thereof increases the cumulative weight of the plant fruits by at least 10%, by at least 20%, by at least 50%, by at least 100%, or by at least 200%, compared to control, and any value and range therebetween. In some embodiments, contacting a plant with an AAA, or by a composition comprising thereof increases the cumulative weight of the plant fruits by 10-50%, 25-75%, 50-150%, 100-250%, 200-400%, 350-500%, 450-750%, or 700-1,000% compared to control. Each possibility represents a separate embodiment of the invention.

In some embodiments, contacting a plant with an AAA, or by a composition comprising thereof increases the plant's fruit average weight by at least 10%, by at least 20%, by at least 50%, by at least 100%, or by at least 200%, compared to control, and any value and range therebetween. In some embodiments, contacting a plant with an AAA, or by a composition comprising thereof increases the plant's fruit average weight by 10-50%, 25-75%, 50-150%, 100-250%, 200-400%, 350-500%, 450-750%, or 700-1,000% compared to control. Each possibility represents a separate embodiment of the invention.

In some embodiments, a plant contacted by an AAA, or by a composition comprising thereof has more flowers, compared to a control plant. As used herein, “more flowers” is measured by total number of flowers per plant, number of stigmas per plant, or any combination thereof.

In some embodiments, contacting a plant with an AAA, or by a composition comprising thereof increases the number of flowers of the plant by at least 10%, by at least 20%, by at least 50%, by at least 100%, or by at least 200%, compared to control, and any value and range therebetween. In some embodiments, contacting a plant with an AAA, or by a composition comprising thereof increases the number of flowers of the plant by 10-50%, 25-75%, 50-150%, 100-250%, 200-400%, 350-500%, 450-750%, or 700-1,000% compared to control. Each possibility represents a separate embodiment of the invention.

As used herein, increased resistance comprises resistance to abiotic stressors. In some embodiments, an abiotic stressor is temperature. In one embodiment, a temperature stressor comprises suboptimal temperatures, wherein suboptimal temperatures are such that a plant performance pre-harvest (e.g., growth, crop, etc.) is reduced compared to the performance pre-harvest in optimal temperature. In some embodiments, the performance of a plant pre-harvest in a suboptimal temperature is at least 5%, at least 10%, at least 20%, at least 40%, at least 50%, at least 75%, at least 90%, or at least 99% reduced compared to the plant performance pre-harvest in an optimal temperature, and any range and value therebetween. In some embodiments, the performance of a plant pre-harvest in a suboptimal temperature is 5-15%, 10-20%, 18-40%, 25-50%, 40-75%, 65-90%, or 90-100% reduced compared to the plant performance in an optimal temperature pre-harvest.

In one embodiment, a temperature stressor comprises suboptimal temperatures, wherein suboptimal temperatures are such that a plant performance post-harvest (e.g., pathogen resistance) is reduced compared to the performance pre-harvest in optimal temperature. In some embodiments, the performance of a plant pre-harvest in a suboptimal temperature is at least 5%, at least 10%, at least 20%, at least 40%, at least 50%, at least 75%, at least 90%, or at least 99% reduced compared to the plant performance pre-harvest in an optimal temperature, and any range and value therebetween. In some embodiments, the performance of a plant pre-harvest in a suboptimal temperature is 5-15%, 10-20%, 18-40%, 25-50%, 40-75%, 65-90%, or 90-100% reduced compared to the plant performance in an optimal temperature pre-harvest.

In some embodiments, a suboptimal temperature is at least ±1° C. from the optimal temperature, at least ±3° C. from the optimal temperature, at least ±5° C. from the optimal temperature, at least ±7° C. from the optimal temperature, at least ±9° C. from the optimal temperature, at least ±10° C. from the optimal temperature, at least ±12° C. from the optimal temperature, at least ±15° C. from the optimal temperature, at least ±20° C. from the optimal temperature, at least ±25° C. from the optimal temperature, at least ±30° C. from the optimal temperature, at least ±40° C. from the optimal temperature, or at least ±50° C. from the optimal temperature, and any value and range therebetween. In some embodiments, a suboptimal temperature is ±1 to ±6° C. from the optimal temperature, ±3 to ±10° C. from the optimal temperature, ±5 to ±15° C. from the optimal temperature, ±7 to ±20° C. from the optimal temperature, ±10 to ±25° C. from the optimal temperature, ±20 to ±35° C. from the optimal temperature, ±30 to ±45° C. from the optimal temperature, or ±40 to ±60° C. from the optimal temperature. Each possibility represents a separate embodiment of the invention. Optimal temperatures adequate for culturing a plant would be apparent to one of ordinary skill in the art.

In some embodiment the suboptimal temperature in lower than the optimal temperature (e.g., cold). In some embodiment the suboptimal temperature in greater than the optimal temperature (e.g., hot). In some embodiments, contacting a plant with an AAA, or by a composition comprising thereof, renders the plant cold-resistant. In some embodiments, contacting a plant with an AAA, or by a composition comprising thereof, renders the plant heat-resistant.

In some embodiments, abiotic stressors are selected from: temperature, salinity, radiation, and draught. In some embodiments, contacting a plant with an AAA, or by a composition comprising thereof, renders the plant heat-resistant, cold-resistant, salinity-resistant, radiation-resistant, draught-resistant, or any combination thereof. As used herein, the terms “resistant” or “tolerant” are interchangeable, and refer to a plant having at least 90%, at least 91%, at least 92%, at least 93%, at least 94%, at least 95%, at least 96%, at least 97%, at least 98%, at least 99% performance (e.g., growth, crop) under suboptimal conditions, as described hereinabove (e.g., temperature, salinity, etc.), compared to the performance of the plant under optimal condition. In some embodiments, a resistant or tolerant plant has a performance of 85-90%, 88-93%, 91-96% or 85-100% under suboptimal conditions compared to the performance of the plant under optimal conditions.

A plant, in some embodiments, is a crop. In another embodiment, a plant is a fruit or a vegetable. In another embodiment, a plant refers either to the harvested parts or to the harvest in a more refined state (husked, shelled, etc.). In another embodiment, a plant is a cultivated plant. In another embodiment, a plant is a fodder crop. In another embodiment, a plant includes horticulture, floriculture and industrial crops.

In another embodiment, a plant belongs to the superfamily Viridiplantae. In another embodiment, a plant is a monocotyledonous. In another embodiment, a plant is a dicotyledonous plant. In another embodiment, a plant is: Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant, eucalyptus, a tree, an ornamental plant, a perennial grass and a forage crop. Alternatively, algae and other non-Viridiplantae can be used for the methods of the present invention.

According to an embodiment, the plant is not an ornamental plant (e.g., African daisy, bellflower, butterfly flower, sunflower, sapphire flower, safflower, rose, poinsettia, monkey-flower, geranium, fuchsia, carnation, dahlia, Araceae, Acanthaceae, Agavaceae, Araliaceae, Asclepiadaceae, Gesneriaceae, Ficus, Polypodiaceae, Vitaceae, rhododendron).

According to some embodiments of the invention, the plant used by the method of the invention is a crop plant such as rice, maize, wheat, barley, peanut, potato, sesame, olive tree, palm oil, banana, soybean, sunflower, canola, sugarcane, alfalfa, millet, leguminosae (bean, pea), flax, lupinus, rapeseed, tobacco, poplar and cotton.

According to some embodiments of the invention the plant is a dicotyledonous plant. According to some embodiments of the invention the plant is a monocotyledonous plant. According to an embodiment, the plant is susceptible to infection by (e.g., any of the above) virus, bacterium, arthropod, insect or nematode. According to an embodiment, the plant is infected by or susceptible for infection or damage by (e.g., any of the above) virus, bacterium, insect, arachnid, or nematode. According to an embodiment, the plant is grown in a habitat infested by (e.g., any of the above) virus, bacteria, insect, nematode, or arachnid (e.g., spider mite).

According to an embodiment, the plant is a cultivated fruit plant. According to an embodiment, the cultivated fruit plant, refers to a plant which fruits are of an economic value. According to an embodiment, the cultivated fruit plant is selected from the group consisting of strawberries, grapes, apples, blueberries, cherries. According to an embodiment, the cultivated fruit plant is not strawberry, peach, apple, orange, lemon, lime, plum, cherry, raspberry, blackberry, tomato, pepper, melon, cucumber, squash, watermelon (when applied to grains or fruits). According to an embodiment, the plant is not a snapdragon, petunia or lisianthus.

As used herein the term, “Phenylalanine” or “Phe” refers to the α-amino acid with the formula C₉H₁₁NO₂. It can be viewed as a benzyl group substituted for the methyl group of alanine, or a phenyl group in place of a terminal hydrogen of alanine. This essential amino acid is classified as neutral, and nonpolar because of the inert and hydrophobic nature of the benzyl side chain. The L-isomer is used to biochemically form proteins, coded for by DNA. The codons for L-phenylalanine are UUC and UUU. Phenylalanine is a precursor for tyrosine; the monoamine neurotransmitters dopamine, norepinephrine (noradrenaline), and epinephrine (adrenaline); and the skin pigment melanin.

As used herein the term, “Tyrosine” or “Tyr” refers to the an amino acid with the formula C₉H₁₁NO₃. It can be viewed as a tyrosyl group substituted for the methyl group of alanine. This non-essential amino acid is classified as neutral, and nonpolar because of the inert and hydrophobic nature of the tyrosyl side chain. The L-isomer is used to biochemically form proteins, coded for by DNA. The codons for L-tyrosine are UAC and UAU.

As used herein the term, “Tryptophan” or “Trp” refers to the α-amino acid with the formula C₁₁H₁₂N₂O₂. This essential amino acid is classified as neutral, and nonpolar because of the inert and hydrophobic nature of the benzyl side chain. The L-isomer is used to biochemically form proteins, coded for by DNA. The codon for Tryptophan is UGG. Tryptophan is a precursor for the neurotransmitter serotonin, the hormone melatonin, and vitamin B3.

An “analog of Phenylalanine” or “Phe” refers to a naturally occurring composition or synthetic analog of Phe which is capable of controlling the pathogen infection in a plant. Without being bound by theory it is suggested that the Phe or analog thereof functions by increasing the shikamate pathway in the plant and specifically production of phenylpropanoids.

According to an embodiment, the Phe analog is a naturally occurring composition. In one embodiment, the use of the term “Phe” includes a composition comprising an effective amount of Phe. According to an embodiment, the Phe analog is aromatic. According to an embodiment, the Phe analog is Tyrosine or a synthetic analog thereof which is capable of controlling a pathogenic infection. Synthetic analogs are commercially available such as from AnaSpec. A non-limiting example list is provided infra. Measures are taken to test for Phyto-toxicity before applying onto the plant. Table 5 below lists some non-limiting examples of Phe and Tyr analogs.

TABLE 5
(2R,3R)-Boc-β-methyl-phenylalanine
(2R,3R)-Boc-β-methyl-phenylalanine
(2R,3R)/(2S,3S)-Racemic-Boc-β-methyl-phenylalanine
(2R,3S)/(2S,3R)-Racemic Boc-β-hydroxyphenylalanine
(2R,3S)/(2S,3R)-Racemic Boc-β-hydroxyphenylalanine
(2R,3S)/(2S,3R)-Racemic Fmoc-β-hydroxyphenylalanine
(2R,3S)/(2S,3R)-Racemic Fmoc-β-hydroxyphenylalanine
(2S,3S)-Boc-β-methyl-phenylalanine
(2S,3S)-Boc-β-methyl-phenylalanine
Boc-α-methyl-3-methoxy-DL-phenylalanine
Boc-α-methyl-3-methoxy-DL-phenylalanine
Boc-α-methyl-D-phenylalanine
Boc-α-methyl-L-phenylalanine
Boc-α-methyl-L-phenylalanine
Boc-β-methyl-DL-phenylalanine
Boc-β-methyl-DL-phenylalanine
Boc-(R)-1,2,3,4-tetrahydroisoquino-line-3-carboxylic acid
Boc-D-Tic-OH
Boc-(R)-1,2,3,4-tetrahydroisoquino-line-3-carboxylic acid
Boc-D-Tic-OH
Boc-(S)-1,2,3,4-tetrahydroisoquinoline-line-3-carboxylic acid
Boc-L-Tic-OH
Boc-(S)-1,2,3,4-tetrahydroisoquinoline-line-3-carboxylic acid
Boc-L-Tic-OH
Boc-2,4-dichloro-D-phenylalanine
Boc-2,4-dichloro-L-phenylalanine
Boc-2-(trifluoromethyl)-D-phenylalanine
Boc-2-(trifluoromethyl)-L-phenylalanine
Boc-2-bromo-D-phenylalanine
Boc-2-bromo-L-phenylalanine
Boc-2-bromo-L-phenylalanine
Boc-2-chloro-D-phenylalanine
Boc-2-chloro-L-phenylalanine
Boc-2-cyano-D-phenylalanine
Boc-2-cyano-L-phenylalanine
Boc-2-cyano-L-phenylalanine
Boc-2-fluoro-D-phenylalanine
Boc-2-fluoro-L-phenylalanine
Boc-2-methyl-D-phenylalanine
Boc-2-methyl-L-phenylalanine
Boc-2-nitro-D-phenylalanine
Boc-2-nitro-L-phenylalanine
Boc-2;4;5-trihydroxy-DL-phenylalanine
Boc-3,4,5-trifluoro-D-phenylalanine
Boc-3,4,5-trifluoro-L-phenylalanine
Boc-3,4-dichloro-D-phenylalanine
Boc-3,4-dichloro-L-phenylalanine
Boc-3,4-difluoro-D-phenylalanine
Boc-3,4-difluoro-L-phenylalanine
Boc-3,4-dihydroxy-L-phenylalanine
Boc-3,4-dihydroxy-L-phenylalanine
Boc-3,4-dimethoxy-L-phenylalanine
Boc-3,5,3’-triiodo-L-thyronine
Boc-3,5-diiodo-D-tyrosine
Boc-3,5-diiodo-L-thyronine
Boc-3,5-diiodo-L-tyrosine
Boc-3-(trifluoromethyl)-D-phenylalanine
Boc-3-(trifluoromethyl)-L-phenylalanine
Boc-3-amino-L-tyrosine
Boc-3-amino-L-tyrosine
Boc-3-bromo-D-phenylalanine
Boc-3-bromo-L-phenylalanine
Boc-3-chloro-D-phenylalanine
Boc-3-chloro-D-phenylalanine
Boc-3-chloro-L-phenylalanine
Boc-3-chloro-L-phenylalanine
Boc-3-chloro-L-tyrosine
Boc-3-cyano-D-phenylalanine
Boc-3-cyano-L-phenylalanine
Boc-3-cyano-L-phenylalanine
Boc-3-fluoro-D-phenylalanine
Boc-3-fluoro-DL-tyrosine
Boc-3-fluoro-DL-tyrosine
Boc-3-fluoro-L-phenylalanine
Boc-3-iodo-D-phenylalanine
Boc-D-Phe(3-I)-OH
Boc-3-iodo-L-phenylalanine
Boc-Phe(3-I)-OH
Boc-3-iodo-L-phenylalanine
Boc-Phe(3-I)-OH
Boc-3-iodo-L-tyrosine
Boc-3-iodo-L-tyrosine
Boc-3-methyl-D-phenylalanine
Boc-3-methyl-L-phenylalanine
Boc-3-nitro-D-phenylalanine
Boc-3-nitro-L-phenylalanine
Boc-3-nitro-L-tyrosine
Boc-3-nitro-L-tyrosine
Boc-4-(Fmoc-aminomethyl)-D-phenylalanine
Boc-4-(Fmoc-aminomethyl)-L-phenylalanine
Boc-4-(trifluoromethyl)-D-phenylalanine
Boc-4-(trifluoromethyl)-L-phenylalanine
Boc-4-amino-D-phenylalanine
Boc-4-amino-D-phenylalanine
Boc-4-amino-L-phenylalanine
Boc-4-amino-L-phenylalanine
Boc-4-benzoyl-D-phenylalanine
Boc-D-Bpa-OH
Boc-4-benzoyl-L-phenylalanine
Boc-L-Bpa-OH
Boc-4-benzoyl-L-phenylalanine
Boc-L-Bpa-OH
Boc-4-bis(2-chloroethyl)amino-L-phenylalanine
Boc-4-bromo-D-phenylalanine
Boc-4-bromo-D-phenylalanine
Boc-4-bromo-L-phenylalanine
Boc-4-bromo-L-phenylalanine
Boc-4-chloro-D-phenylalanine
Boc-4-chloro-L-phenylalanine
Boc-4-chloro-L-phenylalanine
Boc-4-cyano-D-phenylalanine
Boc-4-cyano-L-phenylalanine
Boc-4-cyano-L-phenylalanine
Boc-4-fluoro-D-phenylalanine
Boc-4-fluoro-L-phenylalanine
Boc-4-fluoro-L-phenylalanine
Boc-4-iodo-D-phenylalanine
Boc-4-iodo-L-phenylalanine
Boc-4-iodo-L-phenylalanine
Boc-4-methyl-D-phenylalanine
Boc-4-methyl-L-phenylalanine
Boc-4-nitro-D-phenylalanine
Boc-4-nitro-L-phenylalanine
Boc-5-bromo-2-methoxy-D-phenylalanine
Boc-5-bromo-2-methoxy-L-phenylalanine
Boc-7-hydroxy-(R)-1,2,3,4-tetrahydroisoquinoline-3-carboxylic acid
Boc-hydroxy-D-Tic-OH
Boc-7-hydroxy-(R)-1,2,3,4-tetrahydroisoquinoline-3-carboxylic acid
Boc-hydroxy-D-Tic-OH
Boc-7-hydroxy-(S)-1,2,3,4-tetrahydroisoquinoline-3-carboxylic acid
Boc-hydroxy-Tic-OH
Boc-7-hydroxy-(S)-1,2,3,4-tetrahydroisoquinoline-3-carboxylic acid
Boc-hydroxy-Tic-OH
Boc-D-3,3-diphenylalanine
Boc-D-homophenylalanine
Boc-D-homophenylalanine
Boc-D-pentafluorophenylalanine

Table 6 below lists some non-limiting examples of Trp analogs.

Boc-4-methyl-DL-tryptophan
Boc-6-fluoro-DL-tryptophan
Boc-6-methyl-DL-tryptophan
Boc-DL-7-azatryptophan
Fmoc-(R)-7-azatryptophan
Fmoc-5-benzyloxy-DL-tryptophan
Fmoc-5-bromo-DL-tryptophan
Fmoc-5-chloro-DL-tryptophan
Fmoc-5-hydroxy-L-tryptophan
Fmoc-6-chloro-L-tryptophan
Fmoc-6-methyl-DL-tryptophan
Fmoc-7-methyl-DL-tryptophan
Fmoc-DL-7-azatryptophan

According to an embodiment, the analog is Aspartame. According to an embodiment, the analog is Tyrosine.

According to an embodiment the AAA is at a concentration of 0.01-50 mM, 0.1-50 mM 0.5-50 mM e.g., 0.5-30 mM, 1-50 mM, 5-50 mM, 10-50 mM, 10-30 mM, 5-30 mM, 1-30 mM, 10-20 mM, 0.5-20 mM, 5-20 mM, 1-20 mM, 1-15 mM, 15 to 30 mM or up to 50 mM.

It may be possible to use low concentration (e.g., 0.01-10 mM, 0.01-5 mM, 0.01-1 mM, 0.1-10 mM, 0.1-5 mM, 0.1-1 mM) of AAA, such as Phe, Tyr, Trp, or analogs thereof, especially when used in conjunction with a surfactant or when combinations of AAA and analogs are used (e.g., Phe, Tyr, and Trp).

According to an embodiment the AAA is administered or applied (or contacted) at a concentration of above 2 mM, e.g., above 3 mM, above 4 mM, above 5 mM, above 10 mM, above 15 mM, above 20 mM, above 25 mM, above 30 mM, above 35 mM, above 40 mM, above 45 mM, above 50.

According to an embodiment the AAA is administered at a concentration of 0.1-0.5 mM, 0.4-1 mM, 0.8-1.5 mM, 1-2.5 mM, 2.1-50 mM, 2.5-50 mM, 2.2-8 mM, 2.5-10 mM, 3-10 mM, 2.2-8 mM, 4-50 mM, 4-12 mM, 4-15 mM, 3-50 mM 4-50 mM, 5-50 mM, 10-50 mM, 15-50 mM, 10-30 mM, 5-30 mM, 2.5-30 mM, 10-20 mM, 2.5-20 mM, 5-20 mM, 15 to 30 mM or up to 50 mM. According to one embodiment, at least 2 mM is more than 2 mM.

According to an embodiment, the AAA is administered at a concentration of 2.1-50 mM. According to an embodiment, the AAA, such as Phe or analog thereof is administered at a concentration of 4-12 mM.

According to an embodiment, the AAA, such as Phe or analog thereof is administered at a concentration of 6 mM to 50 mM. According to an embodiment, the Phe or analog thereof is administered at a concentration of 6 mM to 40 mM. According to an embodiment, the Phe or analog thereof is administered at a concentration of 6 mM to 35 mM.

According to an embodiment, the AAA, such as Phe or analog thereof is administered at a concentration that does not cause sedimentation on the plant. According to an embodiment, the Phe or analog thereof is administered at a concentration that does not cause Phe sedimentation on the plant. According to an embodiment, the Phe or analog thereof is administered at a concentration lower than 50 mM.

In one embodiment, the term “treated”, “prevented”, “administered”, “applied”, “controlled” and “contacted” are interchangeable or synonymous.

As used herein “plant” refers to whole plants, a plant tissue, a plant organ, a fruit, a vegetable, an eatable portion of a plant, a grafted plant including seeds, shoots, stems, roots (including tubers), rootstock, scion, and plant cells, tissues, fruit, flower and organs. The plant may be in any form including cuttings and harvested material (e.g., fruit).

The Phe (or analog) can be applied to plants by spraying, dusting, coating, soaking, irrigation, drenching or otherwise treating them with the active ingredients or alternatively, by treating with the active ingredients the plant seeds, the soil around the plant, or the soil, rice pads or the water for hydroponic culture where the seeds are to be sown. The application may be affected either before or after the plant is infected with a pathogen.

According to an embodiment, the regimen is performed such as to control the spread of a pathogen and/or eliminate a pathogen and/or eliminate/reduce/minimize any damage that can be caused by the pathogen.

According to an embodiment, applying comprises pre-harvest applying. According to an embodiment, applying comprises post-harvest applying. According to an embodiment, applying comprises pre-harvest applying and not post-harvest applying. According to an embodiment, applying is multiple administrations of Phe. According to an embodiment, applying comprises post-harvest applying and not pre-harvest applying. According to an embodiment, the plant is at a post-blossom stage. According to an embodiment, the plant is at a blossom stage. According to an embodiment, plant is at a pre-blossom stage. According to an embodiment, applying includes daily applying, weekly applying, bi-weekly applying, monthly applying, bi-monthly applying, seasonally applying etc. In one embodiment, “shelf life” includes storage.

In one embodiment, provided a method for prolonging the shelf life of a plant or a portion thereof, the post-harvest quality of a plant or a portion thereof, or a combination thereof, comprising the steps of: (a) pre-harvest contacting said plant with an effective amount of phenylalanine or an analog thereof; (b) post-harvest contacting said plant with an effective amount of phenylalanine or an analog thereof; or (c) the combination of (a) and (b), thereby prolonging the shelf life of a plant, the post-harvest quality of a plant, or a combination thereof. In one embodiment, provided a method for prolonging the shelf life of a plant or a portion thereof, the post-harvest quality of a plant or a portion thereof, or a combination thereof comprising multiple treatments or contacting episodes as described herein.

In one embodiment, a method for prolonging the shelf life of a plant or a portion thereof, the post-harvest quality of a plant or a portion thereof, includes reducing and/or minimizing postharvest losses that either quantitative or qualitative. In one embodiment, a method for prolonging the shelf life of a plant or a portion thereof, the post-harvest quality of a plant or a portion thereof, includes maintaining the acidity and/or the sugar content within the plant. In one embodiment, a method for prolonging the shelf life of a plant or a portion thereof, the post-harvest quality of a plant or a portion thereof, includes maintaining the taste of the plant. In one embodiment, a quantitative factor includes reduction in weight of the post-harvested plant. In one embodiment, a quantitative factor includes in the number of plants that are discarded due to an improper/insufficient qualitative measure. In one embodiment, postharvest losses that are either quantitative or qualitative are known to one of skill in the art. In one embodiment, a method for prolonging the shelf life of a plant or a portion thereof, the post-harvest quality of a plant or a portion thereof includes reducing the risk of expedited aging resulting from an infection as described herein. In one embodiment, a method for prolonging the shelf life of a plant or a portion thereof, the post-harvest quality of a plant or a portion thereof includes minimizing the reduction of plant water accumulation.

In one embodiment, a method for prolonging the shelf life of a plant or a portion thereof, the post-harvest quality of a plant or a portion thereof is inhibiting, avoiding, treating, overcoming or any combination thereof, a pathogenic infection or an impact of a pathogenic infection. In one embodiment, a method for prolonging the shelf life of a plant or a portion thereof, the post-harvest quality of a plant or a portion thereof, includes postponing or inhibiting triggering of senescence. In one embodiment, a method for prolonging the shelf life of a plant or a portion thereof, the post-harvest quality of a plant or a portion thereof includes inhibiting or reducing the quantity of ethylene bursting from the plant. In one embodiment, a method for prolonging the shelf life of a plant or a portion thereof, the post-harvest quality of a plant or a portion thereof includes avoiding high ethylene concentration and/or inhibition of ethylene synthesis. In one embodiment, a method for prolonging the shelf life of a plant or a portion thereof, the post-harvest quality of a plant or a portion thereof includes avoiding/minimizing injuries to the plant resulting directly or indirectly from a pathogen. In one embodiment, a method for prolonging the shelf life of a plant or a portion thereof, the post-harvest quality of a plant or a portion thereof includes minimizing the risk of flavor, texture, and/or color deterioration resulting directly or indirectly from a pathogen. In one embodiment, a method for prolonging the shelf life of a plant or a portion thereof, the post-harvest quality of a plant or a portion thereof includes minimizing the risk or inhibiting early ripening resulting directly or indirectly from a pathogen. In one embodiment, a method for prolonging the shelf life of a plant or a portion thereof, the post-harvest quality of a plant or a portion thereof includes minimizing the risk of decrease in the amount or concentration of a nutrient within the plant, wherein the decrease is resulting directly or indirectly from a pathogen. In one embodiment, a method for prolonging the shelf life of a plant or a portion thereof, the post-harvest quality of a plant or a portion thereof includes increasing the uniformity of ripening among plants of the same species that were harvested at approximately the same time. In one embodiment, a method for prolonging the shelf life of a plant or a portion thereof, the post-harvest quality of a plant or a portion thereof includes minimizing the risk of decrease in the amount or concentration of a carbohydrate or a sugar within the plant, wherein the decrease is resulting directly or indirectly from a pathogen. In one embodiment, a method for prolonging the shelf life of a plant or a portion thereof, the post-harvest quality of a plant or a portion thereof includes minimizing the risk of decrease in the amount or concentration of an aromatic compound and/or antioxidant within the plant, wherein the decrease is resulting directly or indirectly from a pathogen.

In one embodiment, postharvest treatment with at least 2.5 mM phenylalanine reduced the postharvest decay severity (caused by fungi or bacteria). In one embodiment, postharvest treatment with at least 4 mM phenylalanine reduced the postharvest decay severity (caused by fungi or bacteria). In one embodiment, postharvest treatment comprises postharvest treatment of a fruit. In one embodiment, postharvest treatment comprises postharvest treatment of a crop. In one embodiment, postharvest treatment comprises postharvest treatment of a vegetable. In one embodiment, postharvest treatment comprises postharvest treatment of a plant. In one embodiment, postharvest treatment comprises postharvest treatment of a flower.

When indicated a specific stage, the application can be confined only to this stage or to the recited stage and additionally to another stage. For instance, when indicated applying at blossom, applying can be performed at blossom or blossom+post-blossom (i.e., fruit), or pre-blossom and blossom or pre-blossom and blossom and post blossom. According to an embodiment, applying is post-emergence (of the infection by a pathogen). According to an embodiment, said phenylalanine or analog is formulated in a composition selected from the group consisting of a dip, a spray or a concentrate. According to an embodiment, applying is in the vicinity of or onto the roots, stems, trunk, seed, fruits or leaves of the plant. According to an embodiment, applying is by irrigation, drenching, dipping, soaking, injection, coating or spraying. According to an embodiment, applying is in an open field. According to an embodiment, applying is in a greenhouse. According to an embodiment, applying is in a storage facility (e.g., dark room, refrigerator). According to an embodiment, applying is applying once. According to an embodiment, applying is applying at least twice at any regimen or duration as necessary and/or as described herein.

According to an embodiment, applying comprises repeated application (2 or more applications e.g., every week, seasonal, bi-weekly, bi-monthly etc.). Repeated applications are especially envisaged for field/greenhouse treatments.

According to an embodiment, repeated application comprises weekly, daily, monthly, or bi-monthly administration during blossom, post-blossom, pre-blossom, or any combination thereof. For example, suggested regimen may include but is not limited to, spraying plants in open fields and green house, adding to irrigation of plants grown in the open field, green house and in pots, dipping the whole foliage branch in the solution post-harvest, adding to vase of cut flowers before and/or after harvest and possibly before shipment.

According to an embodiment, the active ingredient (Phe and/or analog) is formulated into a composition where it is mixed with other active ingredients (e.g., fungicides) and/or an agriculturally acceptable carrier.

According to an embodiment such a composition of the invention is shelf stable. The term “shelf stable” refers to a composition of the invention that maintains its activity throughout a given storage period at the recommended conditions (e.g., temperature) and optionally does not separate out into separate phases or develop any offensive odors.

As used herein the term “agriculturally acceptable carrier” refers to a material that facilitates application of a composition of the invention to the intended target, which may be for example a plant, a plant material, compost, earth, surroundings or equipment, or that facilitates storage, transport or handling. Carriers used in compositions for application to plants and plant material are preferably non-phytotoxic or only mildly phytotoxic. A suitable carrier may be a solid, liquid or gas depending on the desired formulation. In one embodiment the carriers include polar liquid carriers such as water, mineral oils and vegetable oils. In one embodiment the carrier enhances the stability of the active ingredient as described herein.

Examples of liquid carriers include but are not limited to water; alcohols, particularly butanol or glycol, as well as their ethers or esters, particularly methylglycol acetate; ketones, particularly acetone, cyclohexanone, methylethyl ketone, methylisobutylketone, or isophorone; petroleum fractions such as paraffinic or aromatic hydrocarbons, particularly xylenes or alkyl naphthalenes; mineral or vegetable oils; aliphatic chlorinated hydrocarbons, particularly trichloroethane or methylene chloride; aromatic chlorinated hydrocarbons, particularly chlorobenzenes; water-soluble or strongly polar solvents such as dimethylformamide, dimethyl sulfoxide, or N-methylpyrrolidone; liquefied gases; or the like or a mixture thereof.

Examples of solid carriers include but are not limited to fillers such as kaolin, bentonite, dolomite, calcium carbonate, talc, powdered magnesia, Fuller's earth, gypsum, diatomaceous earth and China clay. A carrier which provides for slow or delayed release of a compound (Phe or analog) of the invention may also be included in a composition of the invention (especially for the short life cycle pathogens).

In another embodiment, a composition (or active ingredient thereof—Phe or analog) of the invention is applied in an amount capable of inhibiting germination of bacterial spores or bacterial spreading. According to an embodiment, the composition (or active ingredient thereof—Phe or analog) of the invention is applied in an amount capable of reducing the standard concentration advised by a regulatory agency (e.g., FDA, USDA) of commonly used agrotech formulations.

According to an embodiment, the composition (or active ingredient thereof—Phe or analog) of the invention is applied in an amount able to reduce symptoms, spread and/or severity of infection dependent on the pathogen.

As used herein “increasing” or “decreasing” or “reducing” refers to about +/− at least 10%, 20%, 30%, 40%, 50%, 60%, 70%, 80%, 90% or more compared to a control plant in the absence of Phe or analog under identical assay conditions.

Applying may be directly to the plant or to a surface in sufficient vicinity to the plant to control the pathogen infection of the plant. In one embodiment, vicinity is within a distance of 1 meter. In one embodiment, vicinity is within a distance of 0.7 meter. In one embodiment, vicinity is within a distance of 0.5 meter. In one embodiment, vicinity is within a distance of 0.2 meter.

Thus, applying can be to any target surface of a plant or a plant organ to which a compound or composition of the invention may be applied, for example to a plant, plant material including roots, bulbs, fruit, tubers, corms, leaves, flowers, seeds, stems, callus tissue, nuts, grains, fruit, cuttings, root stock, scions, harvested crops including roots, bulbs, tubers, corms, leaves, flowers, seeds, stems, callus tissue, nuts, grains, fruit, cuttings, root stock, scions, or any surface that may contact harvested crops including harvesting equipment, packaging equipment and packaging material.

For surfaces such as harvesting equipment, packaging equipment and packaging material, the compound or composition of the invention is applied before use of the harvesting equipment, packaging equipment or packaging material.

According to an embodiment, the compound or composition of the invention is formulated as a dip, a powder, a spray or a concentrate. According to an embodiment, the formulation comprises a surfactant. According to an embodiment, the surfactant is a cationic surfactant, e.g., benzalkonium chloride, cetylpyridinium chloride. According to an embodiment, the surfactant is an anionic surfactant, e.g., alkyl sulphates, alkyl ethoxylate sulphates. According to an embodiment, the surfactant is a non-ionic surfactant, e.g., Alkyl polyglycoside, Triton X-100, Polyoxyethylene (20) sorbitan monooleate (Tween-80), Silwett L-77. According to an embodiment, the surfactant is Tween-80 or Silwett L-77.

According to an embodiment, the concentration of the surfactant is at least 0.1%. In one embodiment, a composition of the invention may further comprise at least one additional agricultural agent. In an alternative embodiment a composition of the invention may be delivered separately, simultaneously or sequentially with at least one additional agricultural agent.

In one embodiment, a composition of the invention may further comprise at least one additional agricultural agent e.g., fungicide, antibiotic, nematocide and/or insecticide. In an alternative embodiment a composition of the invention may be delivered separately, simultaneously or sequentially with at least one additional agricultural agent.

When formulating a composition of the invention containing an additional agricultural agent or planning delivery of a composition of the invention separately, simultaneously or sequentially with an additional agricultural agent, it may be desirable to assess the degree of phytotoxicity resulting from application of the compositions to plant material over time. This may be assessed according to the methodology well known in the art.

In some embodiments, the AAA can be in a composition with a coating agent, such as but not limited to, a polysaccharide.

Assessment of a composition of the invention or a composition of the invention including or delivered with an additional agricultural agent may include assessment of: (1) Degree of control of the pathogen without stimulating growth of undesirable non-target microbes or harming beneficial organisms. (2) Durability of control. (3) Degree of phytotoxicity and effects on plant development when used repeatedly throughout a portion or the entirety of a growing season. (4) Compatibility with other control products used in the industry.

As described above, the compositions of the present invention may be used alone or in combination with one or more other agricultural agents, including pesticides, insecticides, acaricides, fungicides, bactericides, herbicides, antibiotics, antimicrobials, nematocides, rodenticides, entomopathogens, pheromones, attractants, plant growth regulators, plant hormones, insect growth regulators, chemosterilants, microbial pest control agents, repellents, viruses, phagostimulants, plant nutrients, plant fertilizers and biological control agents. When used in combination with other agricultural agents the administration of the two agents may be separate, simultaneous or sequential. Specific examples of these agricultural agents are known to those skilled in the art, and many are readily commercially available.

Examples of plant nutrients include but are not limited to nitrogen, magnesium, calcium, boron, potassium, copper, iron, phosphorus, manganese, molybdenum, cobalt, boron, copper, silicon, selenium, nickel, aluminum, chromium and zinc. Examples of antibiotics include but are not limited to oxytetracycline and streptomycin. Examples of fungicides include but are not limited to the following classes of fungicides: carboxamides, benzimidazoles, triazoles, hydroxypyridines, dicarboxamides, phenylamides, thiadiazoles, carbamates, cyano-oximes, cinnamic acid derivatives, morpholines, imidazoles, beta-methoxy acrylates and pyridines/pyrimidines. Further examples of fungicides include but are not limited to natural fungicides, organic fungicides, Sulphur-based fungicides, copper/calcium fungicides and elicitors of plant host defenses.

Examples of natural fungicides include but are not limited to whole milk, whey, fatty acids or esterified fatty acids. Examples of organic fungicides include but are not limited to any fungicide which passes an organic certification standard such as biocontrol agents, natural products, elicitors (some of may also be classed as natural products), and Sulphur and copper fungicides (limited to restricted use).

An example of a Sulphur-based fungicide is Kumulus™ DF (BASF, Germany). An example of a copper fungicide is Kocide.RTM. 2000 DF (Griffin Corporation, USA). Examples of elicitors include but are not limited to chitosan, Bion™, BABA (DL-3-amino-n-butanoic acid, beta -aminobutyric acid) and Milsana™ (Western Farm Service, Inc., USA).

In some embodiments non-organic fungicides may be employed. Examples of non-organic fungicides include but are not limited to Bravo™ (for control of powdery mildew (PM) on cucurbits); Supershield™ (Yates, NZ); Topas.RTM. 200EW (for control of PM on grapes and cucurbits); Flint™ (for control of PM on apples and cucurbits); Amistar.RTM. WG (for control of rust and PM on cereals); and Captan™ Dithane™, Euparen™, Rovral™, Scala™, Shirlan™, Switch™ and Teldor™.

Examples of pesticides include but are not limited to azoxystrobin, bitertanol, carboxin, Cu₂O, cymoxanil, cyproconazole, cyprodinil, dichlofluamid, difenoconazole, diniconazole, epoxiconazole, fenpiclonil, fludioxonil, fluquiconazole, flusilazole, flutriafol, furalaxyl, guazatin, hexaconazole, hymexazol, imazalil, imibenconazole, ipconazole, kresoxim-methyl, mancozeb, metalaxyl, R-metalaxyl, metconazole, oxadixyl, pefurazoate, penconazole, pencycuron, prochloraz, propiconazole, pyroquilon, SSF-109, spiroxamine, tebuconazole, thiabendazole, tolifluamid, triazoxide, triadimefon, triadimenol, triflumizole, triticonazole and uniconazole.

Efficacy of compositions of the invention may also be confirmed using field trial assay systems. For example, confirmation of the ability of compositions of the invention to prevent pathogen growth may be obtained by applying a compound or composition of the invention to plant material and then inoculating with a target organism. Efficacy is confirmed by the absence of growth or less growth of the target organism than an untreated control. According to an embodiment the agricultural composition may comprise phenylalanine or an analog thereof and a surfactant (as described herein) for controlling a pathogen infection in a plant, in an open-field and/or a greenhouse.

According to an embodiment the agricultural composition may comprise phenylalanine and tyrosine for controlling a pathogen infection in a plant.

As used herein the term “about” refers to ±10%.

The terms “comprises”, “comprising”, “includes”, “including”, “having” and their conjugates mean “including but not limited to”.

The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure. As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Reference is now made to the following examples, which together with the above descriptions illustrate some embodiments of the invention in a non-limiting fashion.

### Example 1

### Tomato Bacterial Speck

### Background

Causal Agent: Pseudomonas Ssyringae pv. Tomato (Okabe).

Symptoms of bacterial speck are small spots that appear on the leaves of the tomato plant. The spots are small and brown in the center, surrounded by a yellow ring. In severe cases the spots may overlap and look larger and irregular and spread to the fruit.

### Results

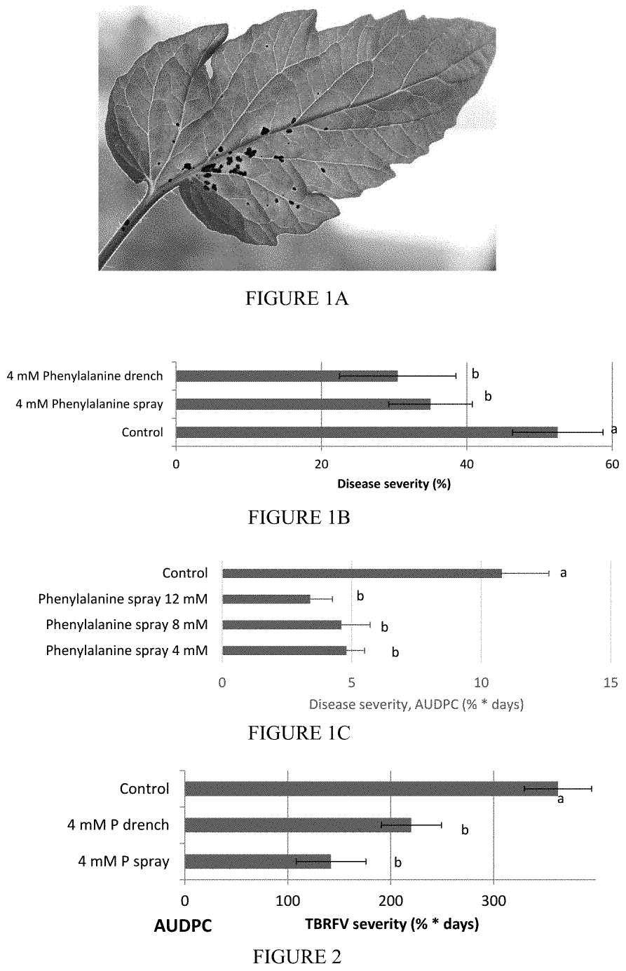

Plants were drenched or sprayed with 4 mM solution of phenylalanine or with water and then incubated at high humidity and temperature of 20-25° C. The plants were naturally infected by a suspension of bacterium cells (10⁷/ml). Phenylalanine and water were applied at 3 days before infection and at the same day of infection. Disease was evaluated according to a 0-100% severity scale where 0=no symptoms (FIGS. 1A-C). FIG. 1B—Severity of bacterial speck on tomato plants treated with drench or spray of 4 mM solution phenylalanine. Disease was evaluated on a 0-100% severity of symptoms coverage 13 days after treatment. Bars=Standard Errors; Columns followed by a different letter are significantly different (P≤0.05). FIG. 1C—Severity of bacterial speck on tomato plants treated with spray of 4-12 mM solution phenylalanine. Disease was evaluated on a 0-100% severity of symptoms coverage and calculation of area under disease progress curve (AUDPC) 14 days after treatment. Columns followed by a different letter are significantly different (P≤0.05).

### Example 2

### Tomato Brown Rugose Fruit Virus TBRFV

### Background

Tomato brown rugose fruit virus (TBRFV) causes symptoms that include a mosaic pattern on leaves accompanied occasionally by narrowing of leaves and yellow spotted fruit. It is a virus that belongs to the Tobamovirus genus.

### Results

Tomato plants were grown in pots in a nonheated greenhouse where temperatures were 17-32° C. Plants were treated and mechanically infected by a suspension of TBRFV from tomato leaves. Phenylalanine and water were applied at 3 days before infection and at the same day of infection. Disease was evaluated on a 0-100% severity of symptoms coverage followed by a calculation of area under disease progress curve (AUDPC) 13 days after treatment. Bars=Standard Errors; Columns followed by a different letter are significantly different (P≤0.05).

### Example 3

### Tuta Absoluta

### Background

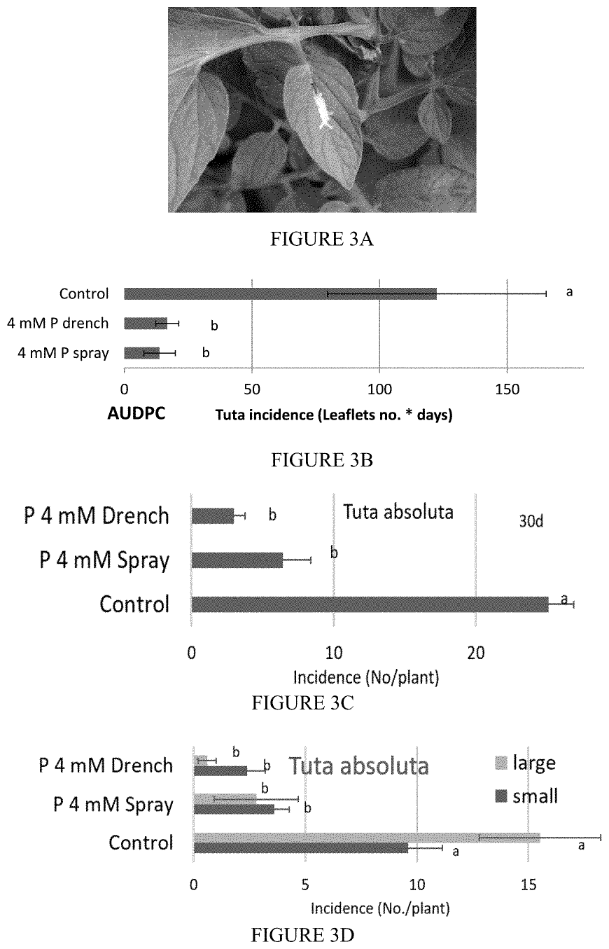

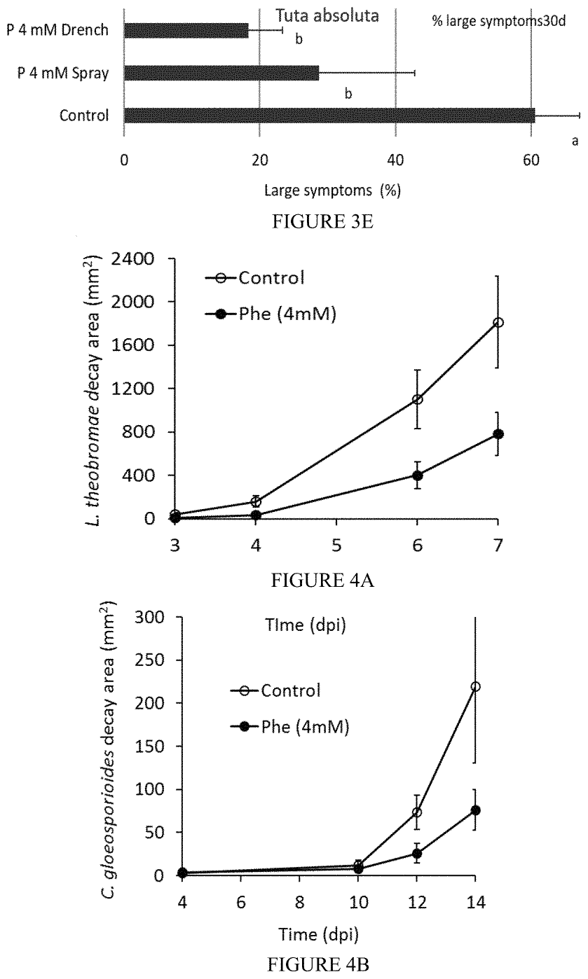

An insect pest that is a moth belonging to the family Gelechiidae. The larva feeds on tomato plants, producing large galleries in leaves. Tomato is the main host plant, but T. absoluta also attacks other crop plants of the including potato, eggplant, pepino, pepper and tobacco and many solanaceous plants. Damage caused by the tomato leaf miner, insect Tuta absoluta on tomato leaflet is shown in FIG. 3A.

### Results

Tomato plants were grown in pots in a nonheated greenhouse where temperatures were 17-32° C. The pest spread in the tomato plants naturally. Damage of the pest was evaluated according to a 0-100% severity scale where 0=no symptoms followed by a calculation of area under disease progress curve (AUDPC) 13 days after treatment. Bars=Standard Errors; Columns followed by a different letter are significantly different (P≤0.05) (FIG. 3B).

Severity of damage of the moth Tuta absoluta leaf miner on tomato leaves treated with 4 mM drench or spray of Phe solution was evaluated: significantly suppressed by each of the treatments (FIG. 3C). The symptom sizes were separated into small (up to 1 cm long) and large. Both kinds of symptoms were suppressed by the Phe spray and the Phe drench treatments (FIG. 3D). The rate (in %) of larger symptoms was reduced by the spray and by the drench treatments meaning an action of resistance towards the development of leaf miner symptoms after the interaction of the miner with the leaf (FIG. 3E).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### Example 4

### Tetranychus Urticae

Tetranychus urticae Koch, the red spider mite (also two-spotted spider mite) is a plant feeding mite of the family Tetranychidae. This mite can feed on tomato, pepper, potato, bean, maize, strawberry and many other crop plants. It sucks the cell contents in leaves causing whitish spots on the leaf surface. Eventually it reduces the photosynthetic ability of the plants, eventually causes leaf mortality and major yield losses.

The red spider mite Tetranychus urticae Koch, (also two-spotted spider mite) occurred naturally on the treated tomato plants. The plants were kept in a greenhouse with 18-28° C. Typical scratching symptoms severity were evaluated on each plant; 0=no infection (all leaves are symptomless) and 100=leaves were fully covered by symptoms.

### Effect of Phe on Red Spider Mite Tetranychus Urticae on Tomato Plants

Severity of damage of the Tetranychus urticae red spider mite on tomato leaves treated with drench or spray of 4 mM Phe solution was significantly suppressed by each of the treatments (FIG. 9).

### Example 5

### Bemisia Tabaci

Background: Bemisia tabaci Gennadius is the silver leaf whitefly (commonly also sweet potato whitefly) is an insect that causes damage to many plant crops by feeding on them and by transmission of virus plant pathogens. It sucks phloem liquid from the leaves, causes whitish dots and secretes honeydew that promotes the development of sooty molds on plant canopies. Many crop plants are affected including tomato, squash, broccoli, cauliflower, cabbage, melon, cotton, carrot, sweet potato, cucumber, pumpkin, and ornamental plants.

### Effect of Phe on Silverleaf White Fly (Bemisia Tabaci) on Tomato Leaves

The insect silver leaf whitefly Bemisia tabaci Gennadius (commonly also sweet potato whitefly) occurred naturally on the treated tomato plants. The plants were kept in a greenhouse with 18-28° C. Incidence of insect individuals' number on the 10^{th} leaf of the tomato plants was counted.

Incidence of the Bemisia tabaci silver leaf whitefly on tomato leaves treated with drench or spray of Phe solution was significantly suppressed by each of the treatments (FIG. 10).

### Example 6

### Oidium Neolycopersici

Oidium neolycopersici Kiss is an obligate parasitic fungus that infects tomato plants. It caused white symptoms on leaves, stems and sepals and eventually can cause the death of the entire canopy of tomato plants. The disease named Oidium powdery mildew spreads by conidia of the fungus and causes damages to tomato plants in greenhouses and open field.

### Effect of Phe on Oidium Neolycopersici Infection on Tomato Plants

Conidia of Oidium neolycopersici were collected from leaves of a diseased plant, suspended in water and sprayed on the tomato plants. The plants were kept in a greenhouse with 18-28° C. Disease severity of Oidium powdery mildew was evaluated on a scale of 0-100% where 100=whole canopy covered by disease symptoms.

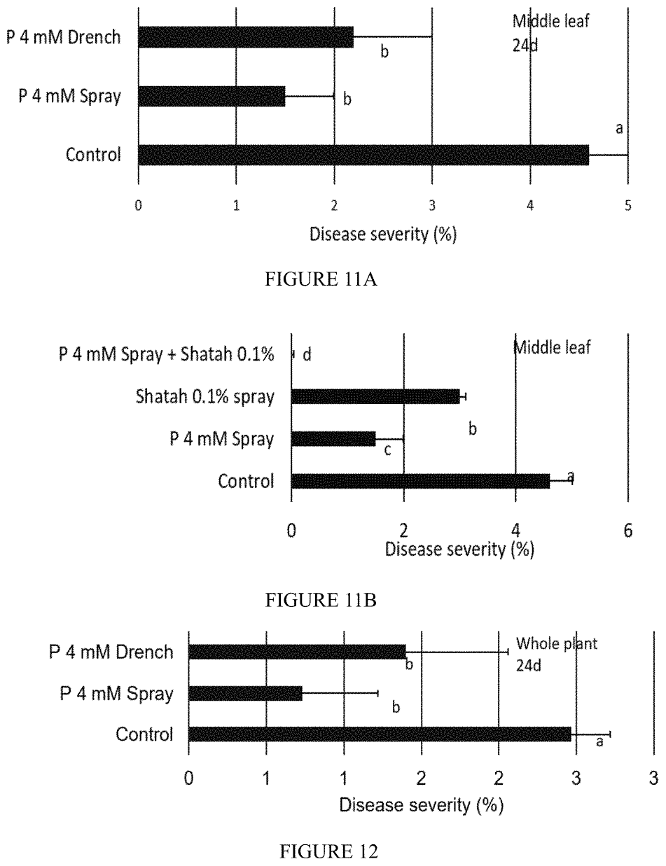

Severity of Oidium powdery mildew on tomato leaves treated with drench or spray of Phe solution was significantly suppressed by each of the treatments (FIG. 11A).

Severity of Oidium powdery mildew on tomato leaves treated with spray of Phe solution, spray of Alkyl Phenol Ethylene Oxide (Shatah 90, Adama Makhteshim) was significantly suppressed by each of the treatments. Combination of the spreader Shatah with Phe spray improved the suppression of the disease (FIG. 11B).

### Example 7

### Oidiopsis Sicula

The effect of Phe on Oidiopsis sicula infection on tomato plants was assessed. Oidiopsis sicula Scalia syn O. taurica (Lév.) Salmon (Teleomorph Leveillula taurica (Lév.) Arnaud) is an obligate parasitic fungus that infects tomato plants and many other agricultural crops like pepper, potato, eggplant. It caused white symptoms on lower parts of the leaves and eventually can cause the death of the entire canopy of tomato plants. The disease named Oidiopsis powdery mildew spreads by conidia of the fungus and causes damages to tomato plants in greenhouses and open field .

Conidia of Oidiopsis sicula were collected from leaves of a diseased plant, suspended in water and sprayed on the tomato plants. The plants were kept in a greenhouse with 18-28° C. Disease severity of Oidiopsis powdery mildew was evaluated on a scale of 0-100% where 100=all leaves covered by disease symptoms.

Severity of Oidiopsis powdery mildew on tomato leaves treated with drench or spray of Phe solution was significantly suppressed by each of the treatments (FIG. 12).

### Example 8

### Tomato Yellow Leaf Curl Virus (TYLCV)

The effect of Phe on TYLCV Begomovirus on tomato plants was assessed. Tomato yellow leaf curl virus (TYLCV) is a DNA virus from the genus Begomovirus. The virus is transmitted by an insect vector the whitefly Bemisia tabaci, (silverleaf whitefly; sweet potato whitefly from the family Aleyrodidae). Plants infected are tomato and also eggplant, potato, tobacco, bean, and pepper. Virus infection causes stunting, reduction of leaf size, upward cupping/curling of leaves, chlorosis on leaves and flowers, and reduction of fruit production.

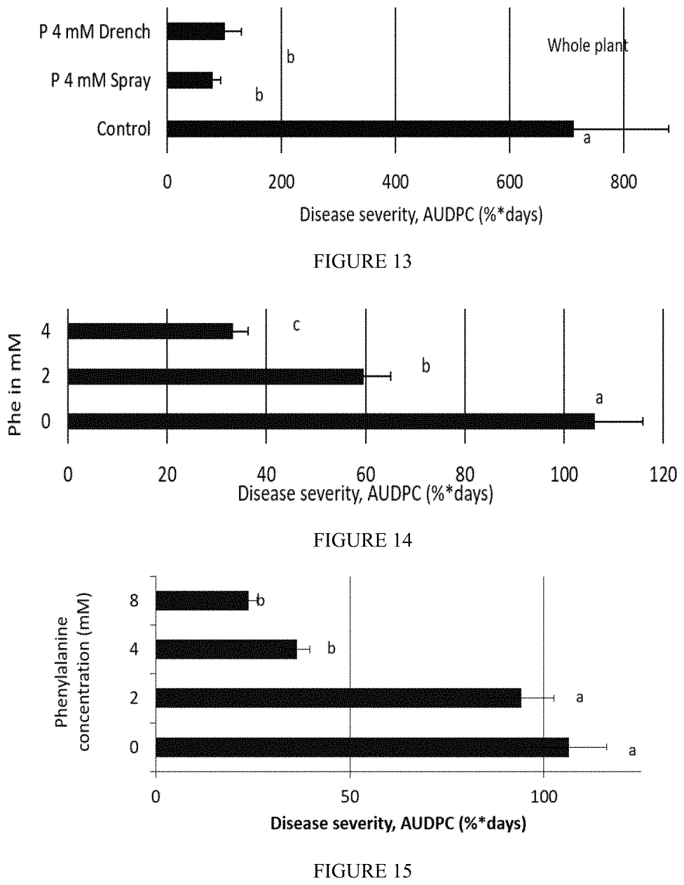

It was found that sprayed Phe solution on tomato plants and drench of in solution to the root zone of tomato plants significantly suppressed the severity of Tomato yellow leaf curl virus on tomato plants canopy (FIG. 13).

### Example 9

### Post-Harvest Protection of Mangos

Postharvest treatments to ‘Shelly’ mango fruits with phenylalanine reduced the decay severity caused by the pathogenic fungi Lasiodiplodia theobromae and Colletotrichum gloeosporioides in comparison to the untreated control fruits (FIG. 4). Lasiodiplodia theobromas and Colletotrichum gloeosporioides are the main pathogens that cause postharvest diseases and fruit loss in subtropical fruit and mango fruit specifically. Interestingly, 2 mM phenylalanine treatment did not suffice while 4 mM phenylalanine substantially inhibited the pathogenic activity of both pathogens.

### Example 10

### Post-Harvest Protection of Avocados

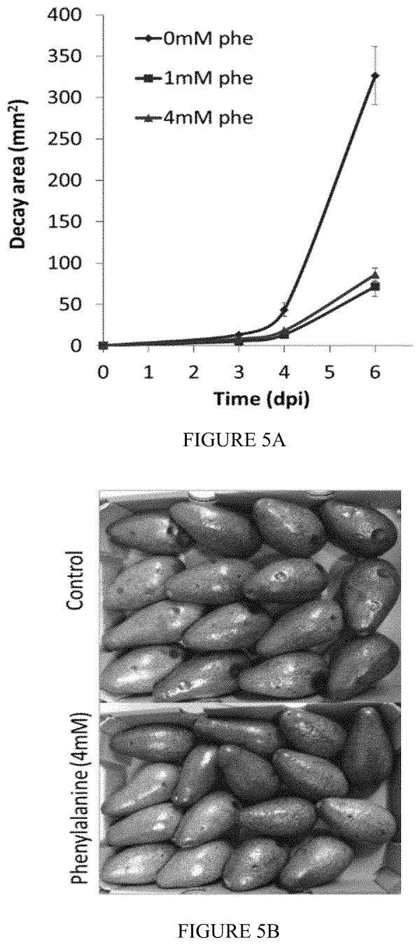

Postharvest treatments to ‘Ettinger’ avocado fruits with 4mM phenylalanine but not 2 mM phenylalanine reduced the decay severity caused by the pathogenic fungi Alternaria alternata in comparison to the untreated control fruits (FIG. 5). Alternaria alternata is one of the main pathogens that cause postharvest diseases and fruit loss in arid and semi-arid areas as Israel.

### Example 11

### Post-Harvest Protection of Clementine

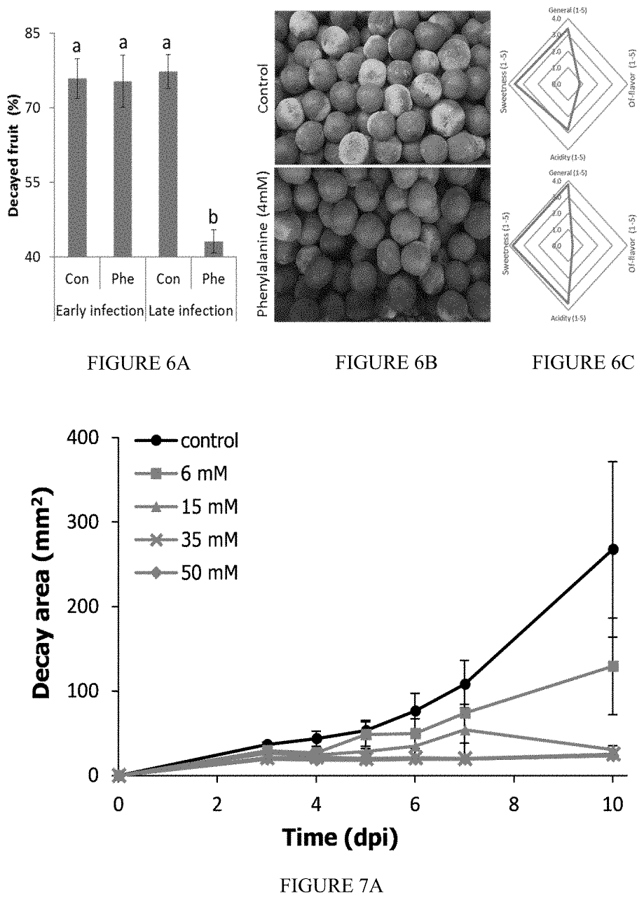

Postharvest treatments to ‘Michal’ clementine fruits with 4mM phenylalanine but not 2 mM phenylalanine reduced the decay severity caused by the pathogenic fungi Penicillium digitatum in comparison to the untreated control fruits (FIG. 6). Penicillium digitatum is the main pathogen that cause postharvest diseases and losses in citrus fruit worldwide. Interestingly, an infection that was implemented directly after the phenylalanine application was not effective, while the later infection that was done two days later was effective in reducing the occurrence of decay caused by Penicillium digitatum (FIG. 6A). This result demonstrates that phenylalanine activates the fruit defense response.

### Example 12

### The Concentration of Phenylalanine is Determinative (1)

The effect of treating petunia plants with increased concentrations of Phe was assessed: plants were either sprayed or drenched with increasing concentration of Phe three days prior to infection with Botrytis and on the day of infection. The infected area was followed for 7-10 days after infection. This experiment demonstrated that 6 mM Phe might not be effective enough in protection against Botrytis.

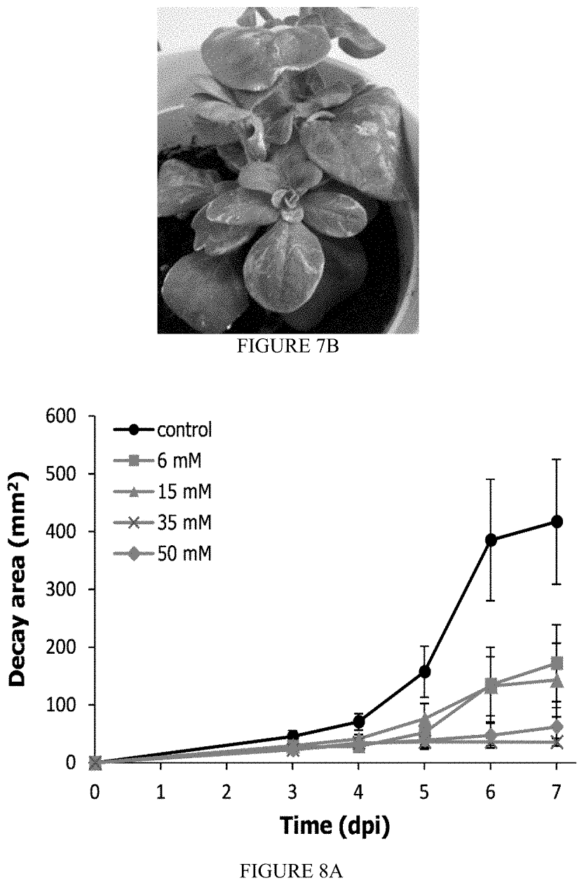

A white sediment (probably Phe salts) accumulated on the leaves of plants sprayed with 50 mM Phe (photo) and to a lesser extent on those sprayed (FIG. 7) with 35 and 15 mM. However, plants treated both with 35 and 50 mM had a much higher resistance to Botrytis.

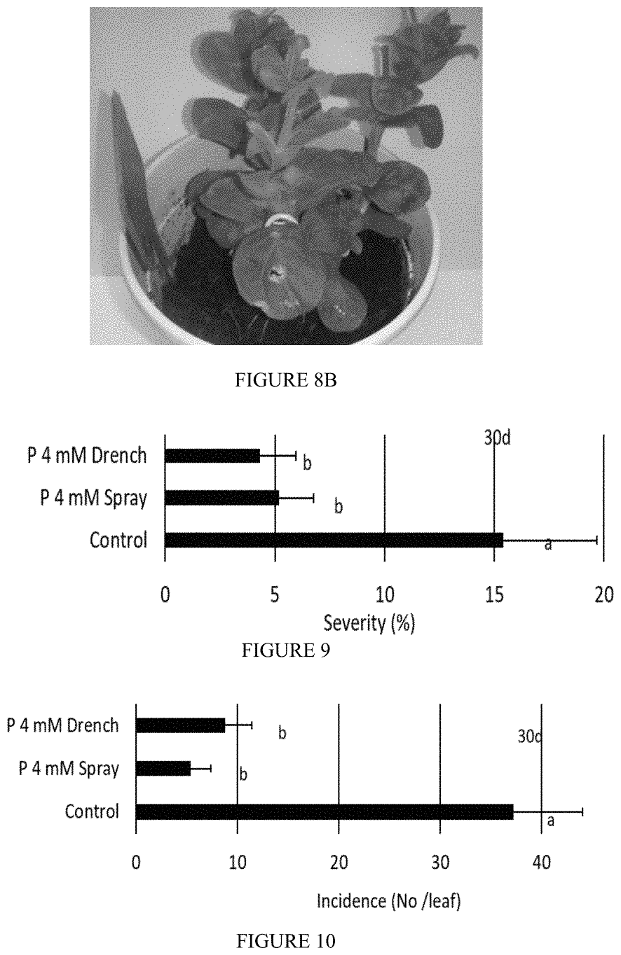

When plants were drenched (FIG. 8) in increasing concentrations of Phe, the high concentrations of 15, 35 and 50 mM were similar to spraying in their effect in increased resistance. However, only the lower leaves of the plants drenched with 50 mM had white sediments. Plants drenched with 15 and 35 mM were both healthy, pathogen free and with no sediments.

### Example 13

### The Concentration of Phenylalanine is Determinative (2)

### Plants

Plants of tomato (Solanum esculentum) and cucumber (Cucumis sativus) were grown from seeds in a nursery and transplanted into 1-liter pots at 40 to 50 days after seeding in an unheated greenhouse. Plants were fertilized proportionally with drippers 2-3 times per day with 5:3:8 NPK fertilizer (irrigation water was planned to have total N, P and K concentrations of 120, 30 and 150 mg/L, respectively; EC 2.2 dS/m), allowing for 25-50% drainage. Plants were maintained at 20 to 30° C. with natural light, and relative humidity of 50-90% in a pest- and disease-free greenhouse during the growth period and then transferred to an area where diseases were allowed to develop following pathogen infection on intact leaves as described below.

### Example 14

### Pre-Harvest AAA-Treatment Inhibits Fruit Decay

Strawberry plants were treated with 1-4 mM solution 3 weeks pre-harvest. Treated plants had substantially reduced amount of rotten fruits (e.g., reduced decay incidence; FIG. 18). This experiment demonstrated that pre-harvest treatment using 1-4 mM Phe is effective in protecting or preventing fruit rotting.

### Example 15

### Post-Harvest AAA-Treatment Inhibits Fruit Decay and Improves Cold Resistance

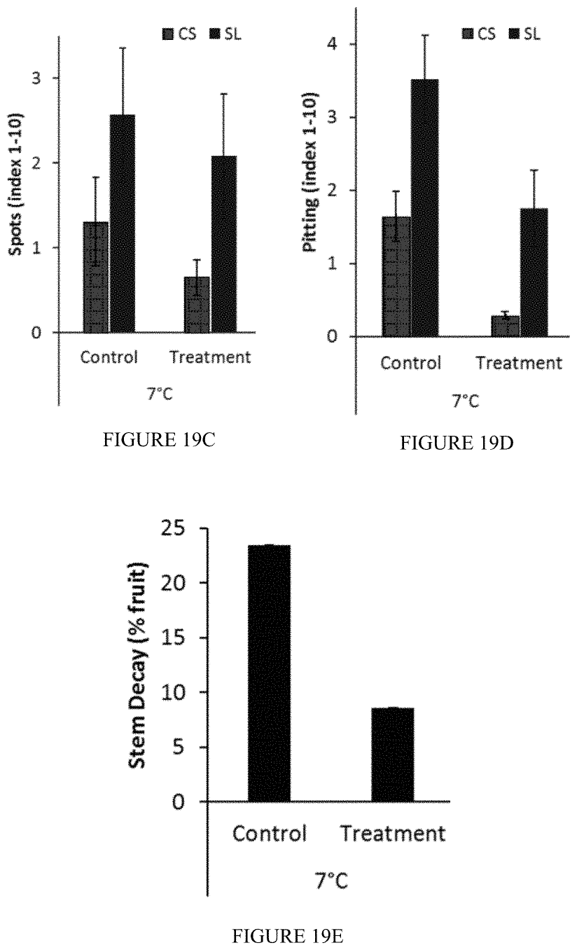

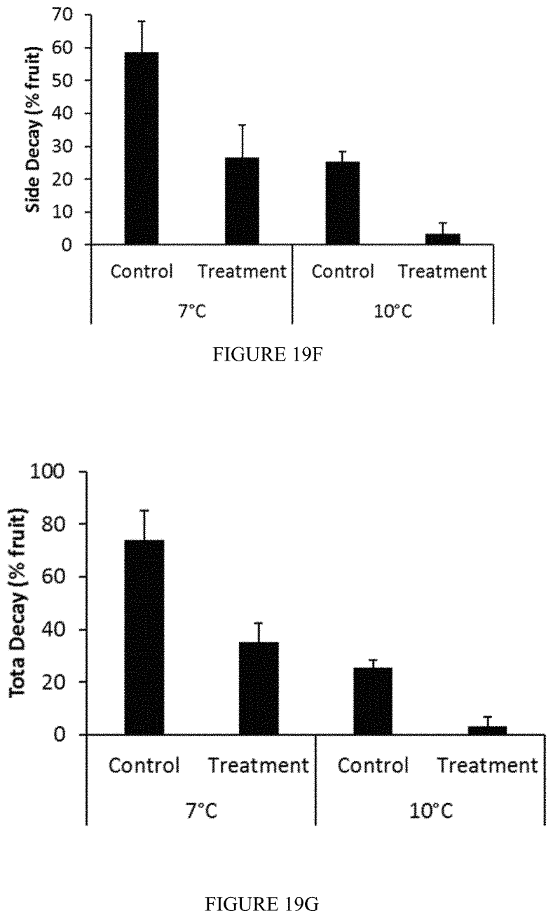

Mango fruits were treated with Phe solution post-harvest. Fruits were stored in either 7° C. or 10° C., and decay was inspected and documented. Post-harvest treatment as described hereinabove reduced stem decay, side decay, and total decay (FIGS. 19E-19G). Furthermore, the AAA treatment improved pesticides resistance under reduced temperature of 7° C. Fruits were both treated with AAA and infested with a pesticide post-harvest. The treated fruits showed substantially reduced levels of black spots and pitting in the presence of either Colletotrichum or Lasiodiplodia under 7° C. (FIGS. 19C-19D). This experiment demonstrated that post-harvest treatment using Phe is effective in protecting or preventing fruit rotting due to pesticides as well as suboptimal temperature.

### Example 16

### AAA Improves Plant Growth Performance Parameters

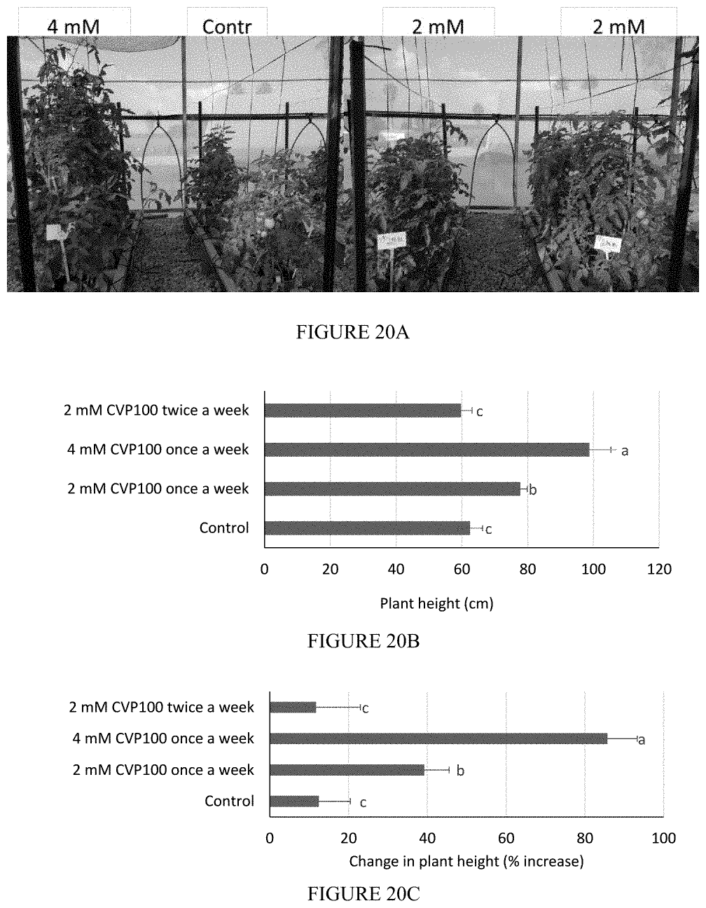

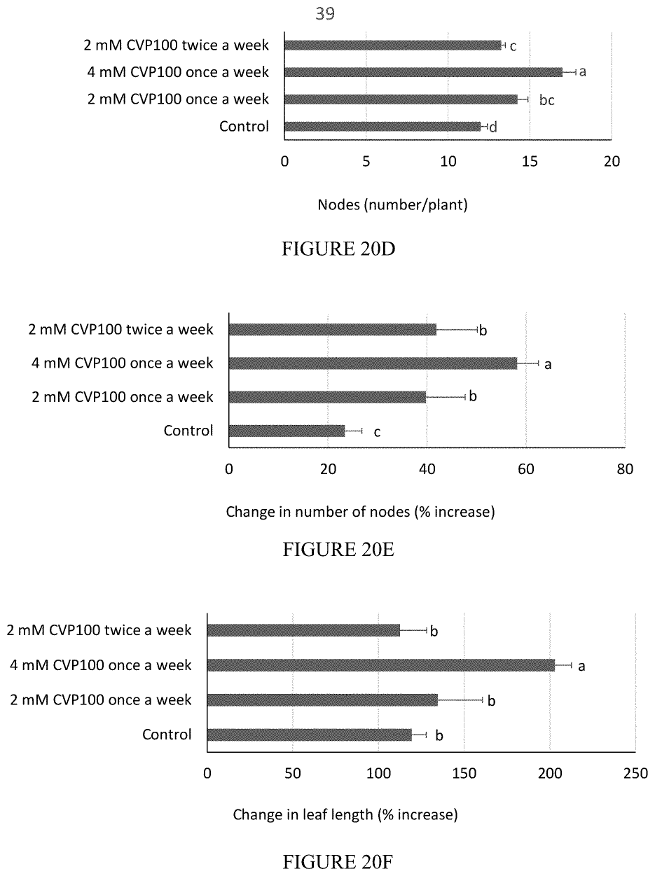

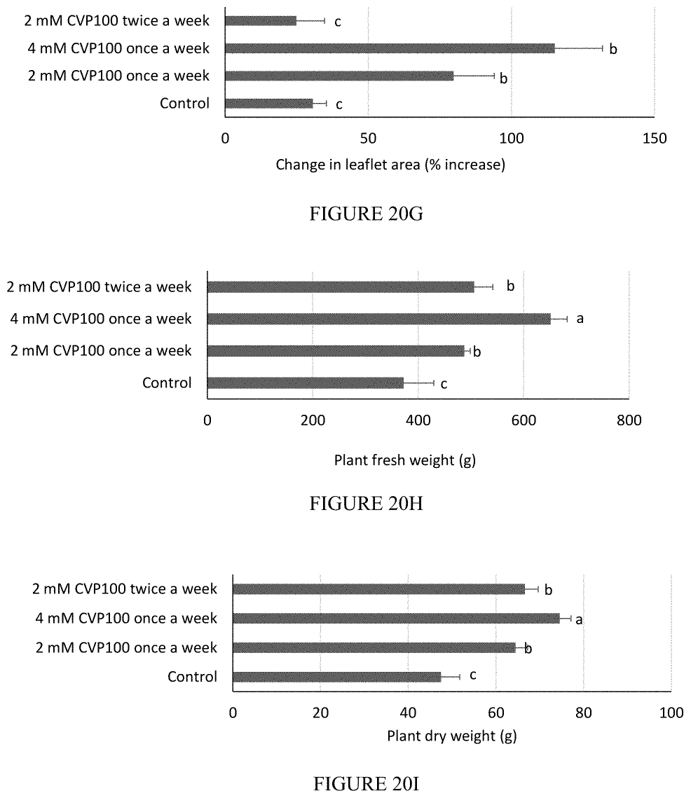

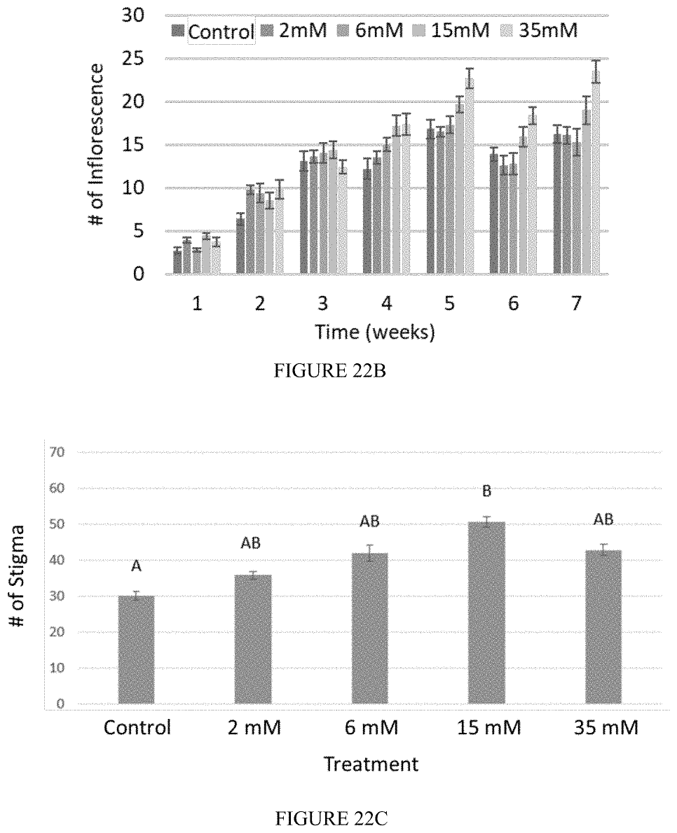

Plant were treated with Phe solution pre-harvest, and growth performance parameters, including total height, total weight (“wet” and dry), number of nodes, leaf length, and leaf area were examined. Plants treated with 2 mM, or 4 mM of Phe, were significantly higher than control (FIGS. 20A-20C). This increase in height was also correlated with the number of nodes on the plant's stem (FIGS. 20D-20E). With respect to leaf physical parameters, control plants had significantly shorter leaves (compared to treated with 4 mM Phe) with a reduced leaf area compared to those of plants treated with Phe (2 mM or 4 mM once a week). Furthermore, the final weights at harvest of treated plants were found to be significantly greater than control, either measured as total “wet” weight or as total dry biomass (FIGS. 20H-20I). In addition, plants treated with the disclosed compounds and composition comprising thereof had more flowers and stigmas compared to control plants (FIGS. 22A-22C). This experiment demonstrated that pre-harvest treating using Phe is effective in increasing plant performance parameters, such as growth-related parameters.

### Example 17

### AAA Improves Crop Yields

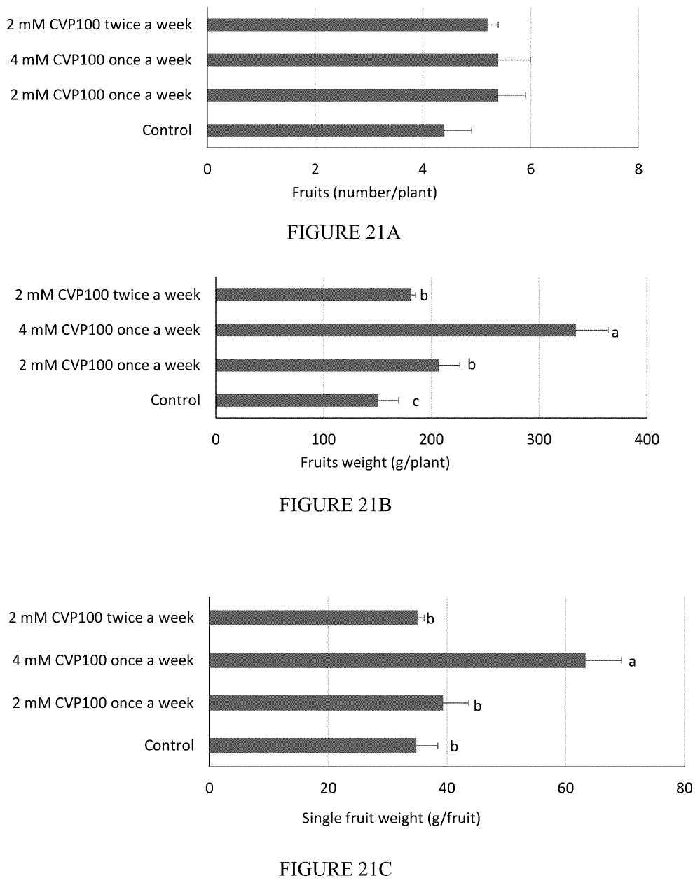

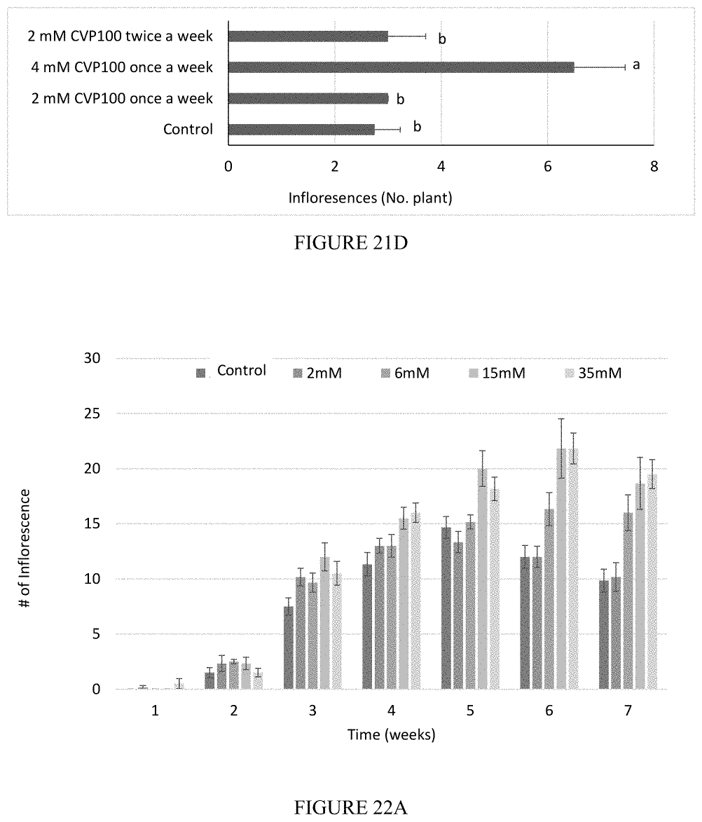

Treating plants with Phe solution significantly increased the total crop yielded (e.g., fruits). With respect to number of fruits, control and treated plants produced comparable amount thereof (FIG. 21A). Nonetheless, treated plant had significantly greater cumulative fruit weight (FIG. 21B), which is also reflected by the significantly greater average weight per fruit in the treated groups (FIG. 21C), compared to control. The production potential of treated plants to produce more than control was exemplified based on the greater number of young fruits (e.g., set fruit), which was recorded in 4 mM-treated plants (FIG. 21D). This experiment demonstrated that pre-harvest treating using Phe is effective in increasing crop yield, by significantly increasing the average fruit weight.

### Statistical Analysis

Treatments in experiments were replicated 5-10 times. Replicates of each treatment were arranged randomly. Disease severity data in percentages were arcsin-transformed before further analysis. Disease severity data were analyzed using ANOVA and Fisher's protected LSD test. Standard errors (SE) of the means were calculated and disease levels were statistically separated (P≤0.05) following a one-way analysis of variance. A control experiment presents the severity of the disease with application of only water without the Phe.

Gray mold inducing pathogen Botrytis cinerea [Pers.:Fr. [Teleomorph: Botryotinia fuckeliana (de Bary) Whetzel] (isolate BcI16; [Swartzberg D. et al., Eur. J. Plant Pathol., 2008, 120:289-297])] was cultured on potato dextrose agar (PDA, Difco, Detroit, Mich.) in 90 mm diam. petri dishes containing 15 ml PDA each and incubated at 20° C. The inoculum was maintained on PDA and transferred every two weeks. Gray mold conidia were harvested from 10 to 14-day-old cultures by agitating 1 cm² of agar bearing mycelium and conidia in a glass tube with tap water. The suspension was then filtered through cheesecloth. The concentration of conidia was determined using a hemocytometer and a light microscope, and adjusted to 5×10⁵ conidia/ml. Since B. cinerea conidia need carbon and phosphate for germination and penetration, 0.1% glucose was added to the final conidial suspension together with 0.1% KH₂PO₄. These supplements have been shown to facilitate germination of B. cinerea conidia and subsequent leaf infection. Plant attached tomato leaves were examined. Whole plants were kept in a humidity chamber at 20±1° C., 97±3% RH, and 1,020 lux light intensity. Plants were infected by spraying the whole plant with 2 ml of a 5×10⁵ conidia/ml suspension or were infected after wounding with a needle and dropping 10 μL drops of conidia suspension prepared as mentioned above. Disease severity was evaluated on each plant using a pictorial key; 0=no infection (all leaves are symptomless) and 100=all leaves are fully covered by gray mold symptoms.

FIG. 14 summarizes the effect of 2-4 mM Phe solution on the severity of tomato gray mold on leaves of tomato plants. The Phe solution was applied by drench to the root zone of the tomato plants at 3 days before and 4 hours before incubation in high humidity in a growth room with temperature of 19-20° C. As shown, 4 mM Phe resulted in significantly better disease control effect than 2 mM.

FIG. 15 summarizes the effect of 2-8 mM solution on the severity of tomato gray mold on leaves of tomato plants. The Phe solution was sprayed the tomato plants at 3 days before and 4 hours before incubation in high humidity in a growth room with temperature of 19-20C. As shown, 4 and 8 mM Phe resulted in significantly better disease control effect than 2 mM.

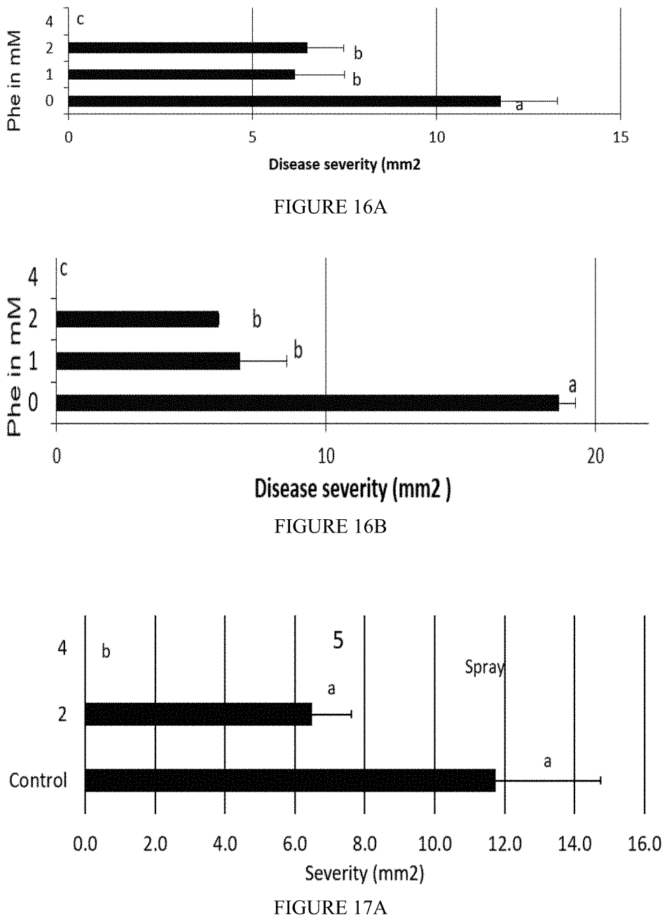

FIG. 16 summarizes the effect of 1-4 mM solution on the severity of tomato gray mold on leaves of tomato plants. The Phe solution was sprayed the tomato plants (upper) or drenched to the tomato plants root zone (lower) at 3 days before and 4 hours before incubation in high humidity in a growth room with temperature of 19-20° C. Infection was made by wounding the leaf tissue and placing a drop of Botrytis cinerea suspension on the wound. As shown, 4 mM Phe resulted in significantly better disease control effect than 2 mM.

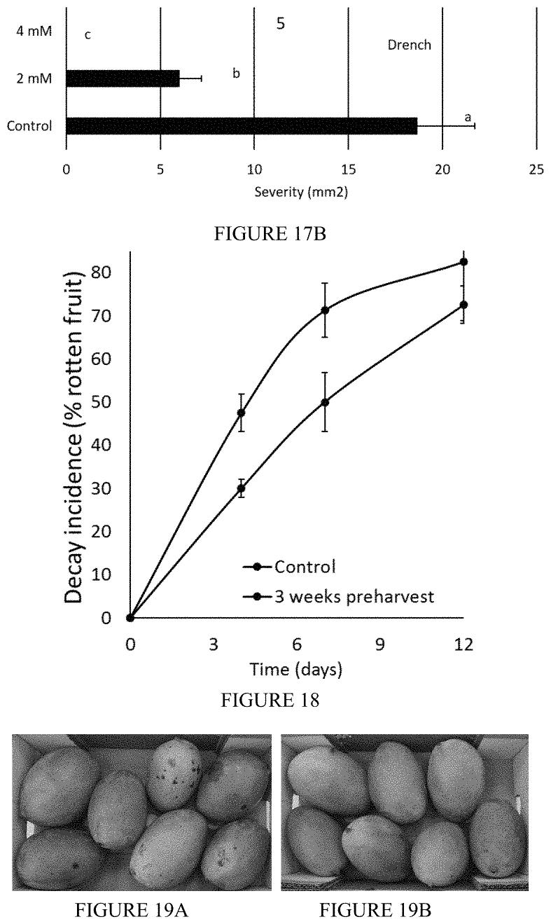

FIG. 17 summarizes the effect of 2-4 mM solution on the severity of tomato gray mold on leaves of tomato plants. The Phe solution was sprayed the tomato plants (upper) or drenched to the tomato plants root zone (lower) at 3 days before and 4 hours before incubation in high humidity in a growth room with temperature of 19-20° C. Infection was made by wounding the leaf tissue and placing a drop of Botrytis cinerea suspension on the wound. As shown, 4 mM Phe resulted in significantly better disease control effect than 2 mM.

### Example 18

### AAA Improves Crop Yields

Flowering is one of the more susceptible organs of the tree. Pathogenic fungi can penetrate during flowering and either infect the flower (e.g., powdery mildew) or endophytically colonize the stem and the fruit without causing any visible symptoms. Those fungi (e.g. Colletotrichum and Lasiodiplodia) become active during abiotic stress or during fruit ripening and cause fruit stem-end rot and anthracnose. Fungicide treatments (Pyrimethanil+Fludioxonil) during orchard flowering reduce postharvest disease.

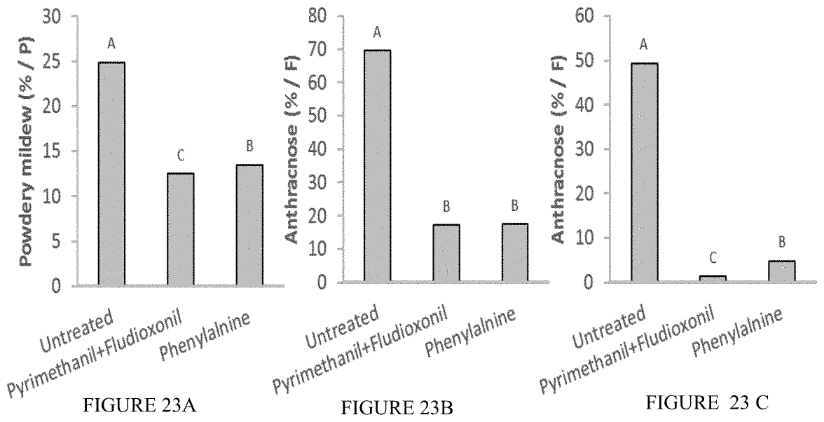

FIG. 23 shows that the application of phenylalanine during orchard flowering reduced various diseases as mango powdery mildew and postharvest fruit disease as anthracnose in both avocado and mango in a similar efficiency to fungicide application.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of controlling a pathogenic infection in a plant, comprising contacting said plant with an effective amount of phenylalanine or an analog thereof at a concentration of at least 2 mM, thereby controlling a pathogenic infection in a plant.

2. The method of claim 1, wherein said pathogenic infection excludes a fungal infection.

3. The method of claim 1, wherein said pathogenic infection is a viral infection, a bacterium infection, a moth infection, an arachnid infection, or any combination thereof.

4. The method of claim 1, wherein said pathogenic infection is a Pseudomonas infection.

5. A method of controlling a viral infection in a plant, comprising contacting the plant with an effective amount of a phenylalanine or an analog thereof, thereby controlling a viral infection in a plant.

6. A method for prolonging the shelf life of a plant, the post-harvest quality of a plant, increasing a plant performance parameter, or a combination thereof, comprising the steps of: (a) pre-harvest contacting said plant with an effective amount of phenylalanine or an analog thereof; (b) post-harvest contacting said plant with an effective amount of phenylalanine or an analog thereof; or (c) the combination of (a) and (b), thereby prolonging the shelf life of a plant, the post-harvest quality of a plant, or a combination thereof.

7. The method of claim 5, wherein said viral infection comprises a tomato brown rugose fruit virus (TBRFV).

8. The method of claim 6, wherein a concentration of said phenylalanine or said analog is above 2 mM.

9. The method of claim 6, wherein said contacting comprises pre-harvest contacting, post-harvest contacting or a combination thereof.

10. The method of claim 6, wherein said contacting is when said plant is at: a post-blossom stage, a blossom stage, a pre-blossom stage, or any combination thereof.

11. The method of claim 6, wherein said plant is a crop.

12. The method of claim 6, wherein said phenylalanine or said analog is formulated in a composition selected from the group consisting of: a dip, a spray, a seed coating, a concentrate, or any combination thereof

13. The method of claim 6, wherein said contacting is contacting in the vicinity of or onto: a root, a stem, a trunk, a seed, a fruit, a flower, a leave, or any combination thereof.

14. The method of claim 6, wherein said contacting is selected from: (i) irrigating, drenching, dipping, soaking, injecting, coating, spraying, or any combination thereof;
and (ii) contacting in a storage facility, a greenhouse, an open field, or any combination thereof.

15. The method of claim 6, wherein said contacting is repeated at least twice.

16. The method of claim 6, wherein said contacting is pre-infection, post infection, or a combination thereof.

17. The method of claim 6, wherein said performance parameter is selected from the group consisting of: plant growth, crop yield, abiotic stress resistance, and any combination thereof.

18. The method of claim 17, wherein said growth comprises one or more parameters selected from the group consisting of: growth rate, plant weight, plant height, leaf length, leaflet area, number of nodes, and distance between adjacent nodes.

19. The method of claim 17, wherein said abiotic stress is suboptimal temperatures.
